(19) United States
(12) Patent Application Publication
(10) Pub. No.: US 2019/0247303 A1
(43) Pub. Date: Aug. 15, 2019

(54) INHALABLE POWDER COMPOSITION COMPRISING IL-13 ANTIBODY

(71) Applicants: VECTURA LIMITED, Chippenham Wiltshire (GB); UCB BIOPHARMA SPRL, Brussels (BE)

(72) Inventors: Frazer Giles MORGAN, Chippenham, Wiltshire (GB); Mark Jonathan MAIN, Chippenham, Wiltshire (GB); Roger PALFRAMAN, Slough, Berkshire (GB); David KIRKE, Slough, Berkshire (GB)

(21) Appl. No.: 16/346,051

(22) PCT Filed: Oct. 31, 2017
(86) PCT No.: PCT/EP2017/077923

§ 371 Date: Apr. 29, 2019

(30) Foreign Application Priority Data
Oct. 31, 2016 (EP) 16196643.7

(51) Int. Cl. A61K 9/00; A61K 9/16; C07K 16/24; A61P 11/06

(52) U.S. Cl. CPC A61K 9/0075; A61K 9/1623; A61K 2039/505; C07K 16/244; A61P 11/06; A61K 9/1617

(57) ABSTRACT

An inhalable powder composition comprises a) an antagonistic antibody which binds human IL-13, b) leucine and c) trehalose. The antibody may comprise a heavy chain, wherein the variable domain of the heavy chain comprises the sequence given in SEQ ID NO:3 and a light chain, wherein the variable domain of the light chain comprises the sequence given in SEQ ID NO:1. Also described is the use of such compositions in the treatment of asthma, as well as inhalers containing such compositions.

The present invention relates to inhalable dry powder antagonistic anti-IL-13 antibody compositions and methods for their preparation and use.

### BACKGROUND OF THE INVENTION

Interleukin 13 (IL-13) is a short chain cytokine produced by activated T cells and has been implicated in a variety of human disorders. For example, elevated levels of IL-13 mRNA and protein have been detected in the lungs of asthmatic patients (Huang, Xiao et al. 1995 J Immunol 155 2688-94). Furthermore, human IL-13 genetic polymorphisms, which also lead to elevated IL-13 levels, have been identified and are associated with asthma and atopy (Heinzmann, Mao et al. 2000 Hum Mol Genet 9 549-59). IL-13 has also been implicated as a key mediator in allergic lung disease, including airway hyperresponsiveness and inflammation.

Therapeutic strategies have therefore been designed to block IL-13 signalling, in particular antibodies that bind to IL-13 or receptors thereof. IL-13 signals by binding to its cell surface receptors, IL-13 receptor alpha 1 (IL-13Rα1) and IL-13 receptor alpha 2 (IL-13Rα2). IL-13Rα1 interacts with IL-13 with low affinity (KD˜10 nM), but following recruitment of the IL-4 receptor alpha (IL-4Rα), a high affinity (KD˜0.4 nM) signalling heterodimeric receptor complex is formed. The IL-13Rα2 on the other hand has a high affinity (KD˜0.25-0.4 nM) for IL-13 and functions as both a decoy receptor negatively regulating IL-13 binding and as a signalling receptor.

Clinical trials for a number of anti-IL-13 antibodies recently completed or currently underway include: Tralokinumab or CAT-354 (a human IgG4 neutralising antibody) for severe uncontrolled asthma; QAX-576 for Idiopathic Pulmonary Fibrosis; Anrukinzumab or IMA-638 (a humanised monoclonal antibody) for asthma; IMA-026 for asthma; CNTO-5825 (a human monoclonal antibody) for asthma; GSK679586 (a humanised IgG,-type monoclonal antibody) for asthma and Lebrikizumab (a humanized monoclonal antibody) for asthma. These trials all detail antibody administration as either intravenous (i.v.) and/or subcutaneous (s.c.).

Hodsman et al. (BJCP, 2012, 75(1): 118-128) discloses a Phase 1, randomized, placebo-controlled dose escalation study of an anti-IL-13 monoclonal antibody, GSK679586, in healthy subjects and mild asthmatics (subjects were not receiving ICS).

Healthy subjects received single intravenous infusions of GSK679586 (0.005, 0.05, 0.5, 2.5, 10 mg kg⁻¹) or placebo and mild intermittent asthmatics received two once monthly intravenous infusions of GSK679586 (2.5, 10, 20 mg kg⁻¹) or placebo. GSK679586 treatment was associated with a reduction in FeNO (fractional exhaled nitric oxide) levels. However, FeNO levels were only examined at 2 weeks (day 41) and 8 weeks (day 84). For example, a mean reduction in FeNO from baseline was observed for 2.5, 10 and 20 mg kg⁻¹ dose groups at day 41 (decreased by 16 ppb [19%], 27 ppb [44%] and 22 ppb [52%], respectively) and day 84 (decreased by 24 ppb [29%], 36 ppb [55%] and 16 ppb [42%], respectively).

Noonan et al (J Allergy Clin Immunol 2013, 132(3): 567-574) discloses a Phase 2, randomized, double-blind, placebo-controlled dose ranging study that evaluated the efficacy and safety of Lebrikizumab (doses investigated: 125, 250, 500 mg) administered subcutaneously for a 12-week treatment period to asthmatics not receiving ICS. Lebrikizumab treatment was associated with a reduction in FeNO levels. However, FeNO levels were only examined after the 12-week treatment period. For example, mean percentage change from baseline in FeNO levels over the 12-week treatment period were −48% (125 mg), −56% (250 mg) and −41% (500 mg).

WO2010/103274 discloses an antagonistic antibody fragment, which binds human IL-13. The antibody fragment is referred to as Ab652. The antibody fragment may be inhaled by nebulisation and formulated as a dry powder.

Wenzel et al (Lancet 2007, 370: 1422-31) discloses a randomised, double-blind, placebo-controlled, parallel group Phase 2a clinical study evaluating nebulised pitrakinra, a recombinant form of the wild-type human interleukin-4 containing two functional mutations at positions 121 (arginine to aspartic acid) and 124 (tyrosine to aspartic acid), or corresponding placebo in asthmatic patients, not receiving ICS, subjected to an allergen challenge. Results illustrated a 4.4% average percentage decrease in FEV₁ in the pitrakinra group compared to an average percentage decrease of 15.9% with placebo. Additionally, treatment with nebulised pitrakinra resulted in a greater reduction in FeNO concentrations compared to placebo.

Antibody formulations stored for extended periods of time are generally considered less stable in the liquid state than in the solid state. To improve the stability of liquid antibody formulations (ensuring protein structure and function is maintained), common practice is to formulate with excipients. However, the use of excipients can still result in protein instability over time (potentially due to the formation of higher order molecular aggregates). Furthermore, liquid state protein formulations typically require refrigeration (e.g. storage between 2° C. and 8° C.) which complicates transportation and distribution, driving up costs.

An alternative option is to formulate solid dry powder protein formulations. One method for preparing relatively stable dry powders containing proteins is lyophilisation. This technique—also referred to as freeze-drying—can however subject proteins to shear stress, freezing stress and dehydration stress which may all cause loss in protein activity. Lyophilised formulations are also less convenient to use and require additional processing steps such as milling prior to use due to bulky cake formations.

Spray drying is another technique used to create solid state protein formulations. It is a one step process used to convert a liquid-based feedstock into a dried powder form by atomizing the feedstock in droplets, into a hot drying-medium, typically air or nitrogen. The process provides enhanced control over particle size, size distribution, particle shape, density, purity and structure. It is therefore a recognised method for formulating dry powder compositions intended for pulmonary delivery (WO96/32149).

WO98/16205 discloses stable glassy state powder compositions. Such compositions generally comprise polyols. The preferred method for preparing the powdered protein composition is spray drying.

WO03/086451 discloses anti-IL-13 immunoglobulin derived proteins including fragments thereof for treating asthma related conditions. Such proteins may be inhaled and delivered by a dry powder inhaler or metered dose inhaler (pMDI). Where delivery is via a pMDI, the formulation may be produced by spray drying.

WO04/060343 discloses antibody-containing particles used to form antibody-containing powders. The prepared spray-dried particles optionally including excipients are administered intravenously following reconstitution.

WO05/079755 discloses IL-13 antagonist powder compositions. Such compositions are prepared by combining IL-13 antagonist, optionally excipient and solvent to form a mixture or solution which is spray-dried. Said compositions may be administered to the lungs of a subject in aerosol form.

WO12/044736 describes respirable dry powders which contain one or more monovalent metal cations. Several powders of the invention were produced by spray drying and powder formulations included excipients such as leucine.

WO13/016754 discloses spray-dried powders comprising biologically active protein or peptide and L-leucine suitable for inhalation. The active peptide or protein is oxytocin and/or an oxytocin derivative.

WO13/173687 describes high concentration monoclonal antibody formulations suitable for subcutaneous administration, where the monoclonal antibody is spray dried and suspended in a non-aqueous suspension vehicle.

WO15/049519 discloses spray-dried powders comprising an inhalable pharmaceutically active protein that have been acoustically blended in a resonant acoustic blender. The powders may also include an excipient material such as trehalose.

Spray drying proteins suitable for inhalation normally require the presence of stabilizing excipients and/or diluents.

One such stabilizing excipient is amorphous trehalose. Amorphous trehalose is well established as an effective bio-stabiliser of labile biomolecules such as proteins. A number of mechanisms underlie the superior stabilising action of trehalose. These include the relatively high glass transition of the anhydrous form (˜117° C.); its high chemical stability; resistance to hydrolysis; and its ability to act as a water substitute during the dehydration of proteins, thus avoiding irreversible changes in protein conformation. In addition, the phase transition of amorphous trehalose to the crystalline dihydrate form has a desiccating action due to sequestering of water previously adsorbed to the amorphous phase. However, the stabilising properties of amorphous trehalose are counterbalanced by a number of distinct disadvantages associated with its adsorption of water. The adsorption of water results in the plasticisation of the amorphous phase and a marked reduction in the glass transition temperature (T_{g}). This sensitivity to water, in the context of the particle surface, promotes powder agglomeration and, unless the powder is stored under controlled conditions, undermines the physical stability of the composition leading to crystallisation, loss of particle integrity and ultimately physical and chemical degradation of the powder, leading to poor aerosolisation. Furthermore, the peptide or protein may become physically and chemically unstable, leading to degradation such as protein aggregation.

Hence a need still remains for the development of trehalose-based powder formulations and methods thereof that provide effective stabilisation of biomolecules e.g. antibodies which bind human IL-13, and which maintain their activity, efficacy and particle size distribution without the requirement of complicated storage conditions, normally associated with liquid antibody formulations.

### SUMMARY OF THE INVENTION

In one aspect of the invention, there is provided an inhalable powder composition comprising a) an antagonistic antibody which binds human IL-13, b) leucine and c) trehalose.

In one aspect of the invention, there are provided inhalable particles comprising a) an antagonistic antibody which binds human IL-13, b) leucine and c) trehalose.

The composition and particles of the invention may be prepared by spray drying. Thus, in another aspect of the invention, there is provided a process for preparing an inhalable dry powder composition, the process comprising the steps of;
(i) preparing a first aqueous solution and/or suspension comprising leucine and trehalose;
(ii) preparing a second aqueous solution and/or suspension comprising an antagonistic antibody which binds human IL-13 and buffer salt;
(iii) mixing the first and second aqueous solutions and/or suspensions from steps (i) and (ii) to form a feedstock solution and/or suspension; and
(iv) spray-drying the feedstock solution and/or suspension from step (iii).

In one aspect of the invention, there are provided spray-dried inhalable particles comprising a) an antagonistic antibody which binds human IL-13, b) leucine and c) trehalose, the particles being obtainable by the process of the present invention.

In one aspect of the invention, there is provided a container comprising an inhalable powder composition comprising a) an antagonistic antibody which binds human IL-13, b) leucine and c) trehalose.

In one aspect of the invention, there is provided a dry powder inhaler comprising an inhalable powder composition comprising a) an antagonistic antibody which binds human IL-13, b) leucine and c) trehalose.

In one aspect of the invention, there is provided a pharmaceutical kit comprising:
(i) an inhalable powder composition, comprising a) an antagonistic antibody which binds human IL-13, b) leucine and c) trehalose, and
(ii) a dry powder inhaler.

In a further aspect of the invention, there is provided an inhalable powder composition comprising a) an antagonistic antibody which binds human IL-13, b) leucine and c) trehalose, for use in the treatment of asthma.

Similarly, the invention provides the use of an inhalable powder composition comprising a) an antagonistic antibody which binds human IL-13, b) leucine and c) trehalose in the manufacture of a medicament for the treatment of asthma.

In a related aspect, the invention provides a method of treatment of asthma in a subject suffering from or susceptible to that condition, which method comprises the administration to the subject of an inhalable powder composition comprising a) an antagonistic antibody which binds human IL-13, b) leucine and c) trehalose.

### BRIEF DESCRIPTION OF THE DRAWINGS

FIG. 1 shows the amino acid sequence for the light chain variable region of an antagonistic IL-13 antibody (CDP7766) (SEQ ID NO: 1).

FIG. 2 shows the amino acid sequence for the light chain variable region and constant region of an antagonistic IL-13 antibody (CDP7766) (SEQ ID NO: 2).

FIG. 3 shows the amino acid sequence for the heavy chain variable region of an antagonistic IL-13 antibody (CDP7766) (SEQ ID NO: 3).

FIG. 4 shows the amino acid sequence for the heavy chain variable and constant region of an antagonistic IL-13 antibody (CDP7766) (SEQ ID NO: 4).

FIG. 5 shows a table illustrating the VR942 0.5 mg inhalation powder mean particle size distribution (PSD); mean glass transition (T_{g}) by DSC; mean moisture content (KF) and mean potency by IL-13 binding activity (ELISA) compared against reference standard up to 24 months in unit blisters at various environmental conditions.

FIG. 6 shows a table illustrating the VR942 5 mg inhalation powder mean particle size distribution (PSD); mean glass transition (T_{g}) by DSC; mean moisture content (KF) and mean potency by IL-13 binding activity (ELISA) compared against reference standard up to 24 months in unit blisters at various environmental conditions.

FIG. 7a shows a table illustrating the demographic characteristics of the placebo and active treatment groups for a clinical trial designated VR942/1/001, Part 1—healthy volunteers (SAD).

FIG. 7b shows a table illustrating the demographic characteristics of the placebo and active treatment groups for the clinical trial VR942/1/001, Part 2—mild asthmatics (MAD).

FIG. 8a shows a table summarising the number of adverse events (AEs), treatment emergent adverse events (TEAEs) and TEAEs related to treatment recorded for placebo and active treatment groups for the clinical trial VR942/1/001, Part 1—healthy volunteers (SAD).

FIG. 8b shows a table summarising the number of adverse events (AEs), treatment emergent adverse events (TEAEs), TEAEs related to treatment and TEAEs related to device recorded for placebo and active treatment groups for the clinical trial VR942/1/001, Part 2—mild asthmatics (MAD).

FIG. 9 shows graphical representation of the mean FeNO percentage (%) reduction from baseline for placebo and active treatment groups for the clinical trial VR942/1/001, Part 2—mild asthmatics.

FIG. 10 shows graphical representation of the mean absolute FeNO parts per billion (ppb) reduction from baseline parts per billion (ppb) for placebo and active treatment groups for the clinical trial VR942/1/001, Part 2—mild asthmatics.

FIG. 11 shows a table summarising FeNO responders. Responders were defined as subjects achieving at least 10 ppb reduction given a FeNO at baseline less than 50 ppb or achieving a 30% reduction given a FeNO at baseline of at least 50 ppb.

FIG. 12 shows graphical representation of mean FEV₁ (L) increase from baseline for placebo and active treatment groups for the clinical trial VR942/1/001, Part 2—mild asthmatics (associated data is summarised in FIG. 16).

FIG. 13a shows an immunogenicity summary for placebo and active treatment groups for the clinical trial VR942/1/001, Part 11'healthy volunteers (SAD).

FIG. 13b shows an immunogenicity summary for placebo and active treatment groups for the clinical trial VR942/1/001, Part 2—mild asthmatics (MAD).

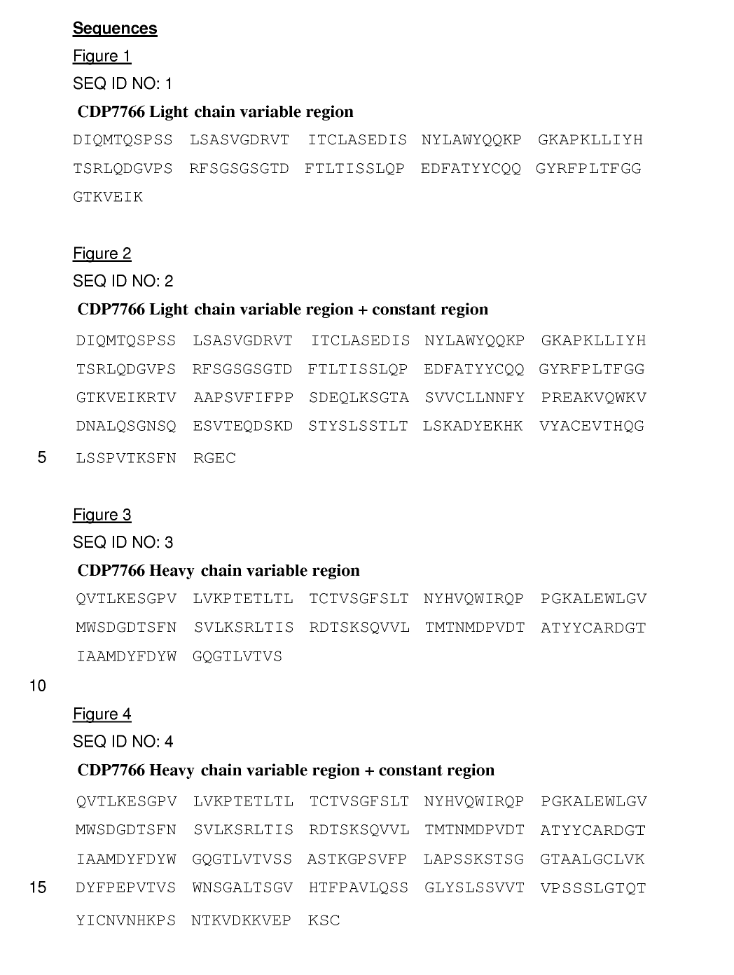

### APPENDIX

FIG. 14 shows a table summarising the FeNO percentage (%) reduction from baseline for placebo and active treatment groups for the clinical trial VR942/1/001, Part 2—mild asthmatics.

FIG. 15 shows a table summarising the FeNO (ppb) absolute reduction from baseline for placebo and active treatment groups for the clinical trial VR942/1/001, Part 2—mild asthmatics.

FIG. 16 shows a table summarising the FEV₁ (L) increase from baseline for placebo and active treatment groups for the clinical trial VR942/1/001, Part 2—mild asthmatics.

### DEFINITIONS

It will be understood that particular embodiments described herein are shown by way of illustration and not as limitations of the invention. The principal features of this invention can be employed in various embodiments without departing from the scope of the invention. Those skilled in the art will recognise, or be able to ascertain using no more than routine study, numerous equivalents to the specific procedures described herein. Such equivalents are considered to be within the scope of this invention and are covered by the claims.

The term “Dry Powder” refers to compositions that comprise inhalable dry particles that are readily dispersible in an inhalation device to form an aerosol. Preferably the inhalable dry powders contain water below about 10%, usually below 5% or below 3% by weight of the inhalable dry particles.

The term “Antagonistic Antibody Which Binds Human IL-13” refers to a complete antibody molecule having full length heavy and light chains or a fragment thereof, such as a Fab, modified Fab′, Fab′, F(ab′)₂, Fv, VH, VL or scFv fragment that is capable of inhibiting and/or neutralising the biological signalling activity of IL-13, for example, by blocking binding or substantially reducing binding of IL-13 to IL-13 receptor and thus inhibiting the activation of the receptor.

The term “CDP7766” refers to a biological drug substance, which is an antagonistic anti-human interleukin (IL)-13 monoclonal antibody fragment (Fab′), described as Ab652 in WO2010/103274, the text of which is incorporated by reference herein.

The term “VR942 Drug Product” refers to a powder drug product for inhalation which includes the biological CDP7766 drug substance, trehalose dihydrate and L-leucine.

The term “Sodium Phosphate”, (chemical structure being NaH₂PO₄) is also referred to as monosodium phosphate, anhydrous monobasic sodium phosphate and sodium di-hydrogen phosphate, all of which may be used interchangeably.

The term “Leucine” is intended to encompass salt forms or counterion formulations of leucine as well as isolated stereoisomers (e.g. D-leucine or L-leucine) and mixtures of stereoisomers. Derivatives and intermediates of leucine are also encompassed.

The term “Inhalation” or “Inhalable” refers to particles that are suitable for pulmonary administration. Such particles typically have a mean aerodynamic particle size of less than 10 μm, more preferably less than 5 μm and most preferably less than 3.5 μm.

The term “d₁₀” refers to the size in microns below which 10% of the particles reside on a volume basis.

The term “d₅₀” refers to the size in microns above or below which 50% of the particles reside on a volume basis.

The term “d₉₀” refers to the size in microns below which 90% of the particles reside on a volume basis.

The term “Glass Transition Temperature”, which is represented by the symbol T_{g}, refers to the temperature at which a composition changes from a glassy or vitreous state to a syrup or rubbery state. T_{g} is generally determined using differential scanning calorimetry (DSC).

The term “Container” refers to either a bulk storage container, such as a multi-dose reservoir for a dry powder inhaler, or unit dose containers such as a capsule or a blister.

The capsule may be formed from various materials e.g. gelatine, cellulose derivatives such as hydroxypropyl methylcellulose (HPMC) or hydroxypropylcellulose (HPC), starch, starch derivatives, chitosan or synthetic plastics, while the blister may be provided in the form of a blister pack or blister strip.

The term “Passive Device” refers to a dry powder inhaler device (either unit dose or multi-dose) in which a patient's breath is the only source of gas which provides the motive force in the device.

The term “Active Device” refers to a dry powder inhaler device (either unit dose or multi-dose) in which a source of compressed gas or an alternative energy source is used to provide the motive force in the device.

The term “therapeutically effective amount” refers to an amount of protein or peptide required to provide a desired therapeutic effect.

The term “FeNO” refers to fraction of exhaled nitric oxide (NO), and is a pharmacodynamic biomarker expressed in parts per billion (ppb). NO is produced by the human lung and is present in the exhaled breath and can be measured using for example, a NIOX MINO® analyser.

The term “FEV₁” refers to forced expiratory volume in one second, which is a type of pulmonary function test that measures the volume of gas that can be forcibly exhaled in one second.

The term “Single Ascending Dose” (SAD) refers to subjects being given a single dose and where different subject groups will receive ascending single doses in sequence.

The term “Multiple Ascending Dose” (MAD) refers to subjects being given several doses (or for example one dose per day), and different subject groups will get higher doses in increasing sequence.

The term “Nominal Dose” (ND) refers to the amount of active present in the container (also termed “Metered Dose” herein). Active is for example the biological drug substance, CDP7766.

The term “Delivered Dose” (DD) refers to the amount of active released from the container and available for inhalation. Active is for example the biological drug substance, CDP7766.

The term “Respiratory Dose” also referred to as “Fine Particle Mass” (FPM) refers to the amount of active delivered from the container that can potentially reach the lungs (particle size of <5 μm). Active is for example the biological drug substance, CDP7766.

The term “Tap Density,” also known as tapped bulk density or tapped density, refers to the maximum packing density of a powder achieved under the influence of well-defined externally applied forces.

The term “subject” or “subjects” include references to mammalian (e.g. human) subjects.

### GENERAL STATEMENTS

As used in this specification and the claim(s), the use of the word “a” or “an” when used in conjunction with the term “comprising” in the claim(s) and/or the specification may mean “one”, but it is also consistent with the meaning of “one or more”, “at least one”, and “one or more than one”. The use of the term “or” in the claim(s) is used to mean “and/or” unless explicitly indicated to refer to alternatives only or the alternatives are mutually exclusive, although the disclosure supports a definition that refers to only alternatives and “and/or”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “any combinations thereof” as used herein refers to all permutations and mixtures of the listed items preceding the term. For example, “A, B, C, or any combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are mixtures that contain repeats of one or more items or terms, such as BB, AAA, BBC, AAABCCCC, CBBAAA, CABABB, and so forth. The skilled artisan will understand that typically there is no limit on the number of items or terms in any mixture, unless otherwise apparent from the context.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

### DETAILED DESCRIPTION OF THE INVENTION

In one aspect of the invention, there is provided an inhalable powder composition comprising a) an antagonistic antibody which binds human IL-13, b) leucine and c) trehalose.

In one embodiment of the invention, the antibody is selected from the group consisting of: a complete antibody molecule having full length heavy and light chains or a fragment thereof, such as a Fab, modified Fab′, Fab′, F(ab′)₂, Fv, VH, VL or scFv fragment.

In a further embodiment of the invention, the antibody comprises a heavy chain, wherein the variable domain of the heavy chain comprises the sequence given in SEQ ID NO:3 and, additionally comprises a light chain, wherein the variable domain of the light chain comprises the sequence given in SEQ ID NO:1.

In an embodiment of the invention, the antibody is CDP7766.

In some embodiments of the invention, the antibody comprises a light chain that has at least 60% homology, identity or similarity to the sequence given in SEQ ID NO:1, or at least 70%, at least 80%, at least 90%, at least 95% or at least 98% homology, identity or similarity.

In some embodiments of the invention, the antibody comprises a heavy chain that has at least 60% homology, identity or similarity to the sequence given in SEQ ID NO:3, or at least 70%, at least 80%, at least 90%, at least 95% or at least 98% homology, identity or similarity.

In one embodiment of the invention, the antibody is present in an amount less than or equal to about 40% by weight of the dry weight of the powder composition, for example less than or equal to about 30%, such as less than or equal to about 20% or less than or equal to about 10% or less than or equal to about 4%, or less than or equal to about 3%, or less than or equal to about 2%, or less than or equal to about 1% or less than or equal to about 0.5%. The antibody may be present in an amount greater than or equal to about 0.5%, about 1%, about 2%, about 3% or about 4% by weight of the dry weight of the powder composition. For example, in one embodiment the antibody is present in an amount of from about 0.5% to about 40%, or about 1% to about 40%, or about 2% to about 40% or 3% to about 40% or about 4% to about 40% by weight of the dry weight of the composition. For example, in one embodiment the antibody is present in an amount of from about 10% to about 40% or about 20% to about 40% or about 30% to about 40% by weight of the dry weight of the composition.

In one embodiment of the invention, the leucine is present in an amount less than or equal to about 25% by weight of the dry weight of the powder composition, for example less than or equal to about 20%, such as less than or equal to about 15% or less than or equal to about 10%, or less than or equal to about 5%. The leucine may be present in an amount of greater than or equal to about 5% or about 10% by weight of the dry weight of the powder composition. For example, in one embodiment the leucine is present in an amount of from about 5% to about 25% by weight of the dry weight of the powder composition, more preferably from about 10% to about 20% by weight of the dry weight of the powder composition.

In another embodiment of the invention, the antibody is present in less than or equal to about 40%, about 30%, about 20% or about 4% by weight of the dry weight of the powder composition and the leucine is present in less than or equal to about 20%, such as less than or equal to 15% or less than or equal to about 10% by weight of the dry weight of the powder composition, and typically more than 5% by weight of the dry weight of the powder composition.

In one embodiment of the invention, the antibody is present in an amount of from about 4% to about 40% by weight of the dry weight of the composition and the leucine is present in an amount of from about 10% to about 20% by weight of the dry weight of the composition

In one embodiment of the invention, the trehalose is present in an amount less than or equal to about 90% by weight of the dry weight of the powder composition, for example less than or equal to about 80%, less than or equal to about 75%, less than or equal to about 70%, less than or equal to about 65%, less than or equal to about 60%, less than or equal to about 55%, less than or equal to about 50%, less than or equal to about 45%, or less than or equal to about 40%, or less than or equal to about 30% or less than or equal to about 20%. The trehalose may be present in an amount of greater than or equal to about 40% or about 55% by weight of the dry weight of the powder composition. In one embodiment the trehalose is present in an amount of from about 40% to about 90% by weight of the dry weight of the composition, or from about 55% to about 65% by weight of the dry weight of the composition.

In another embodiment of the invention, the antibody is present in an amount less than or equal to about 40% by weight of the dry weight of the powder composition, the leucine is present in an amount of about 10% or from about 5% to about 25% by weight of the dry weight of the powder composition and the trehalose is present in an amount of about 45% or from about 35% to about 50% by weight of the dry weight of the powder composition.

In another embodiment of the invention, the antibody is present in less than or equal to about 30% by weight of the dry weight of the powder composition and the leucine is present in an amount of about 10% or from about 5% to about 25% by weight of the dry weight of the powder composition and the trehalose is present in an amount of about 35% or from about 25% to about 40% by weight of the dry weight of the powder composition.

In another embodiment of the invention, the antibody is present in less than or equal to about 20% by weight of the dry weight of the powder composition and the leucine is present in an amount of about 10% or from about 5% to about 25% by weight of the dry weight of the powder composition and the trehalose is present in an amount of about 67% or from 57% to about 70% by weight of the dry weight of the powder composition.

In yet another embodiment of the invention, the antibody is present in less than or equal to about 4% by weight of the dry weight of the powder composition and the leucine is present in an amount of about 10% or from about 5% to about 25% by weight of the dry weight of the powder composition and the trehalose is present in an amount of about 85% or from about 75% to about 90% by weight of the dry weight of the powder composition.

In one embodiment of the invention the trehalose is present as amorphous trehalose. For example, in one embodiment the trehalose forms an amorphous matrix with the antibody.

In one embodiment of the invention, the composition further comprises buffer salts such as phosphate buffered saline (PBS). PBS buffer components include sodium chloride (NaCl) and a phosphate salt such as sodium phosphate (Na₂HPO₄). In one embodiment, the total buffer salts are present in an amount of less than or equal to about 7.5% by weight of the dry weight of the powder composition, for example less than or equal to about 6% or less than or equal to about 5.3% or less than or equal to about 4% or less than or equal to about 3% or less than or equal to about 2.7% or less than or equal to about 2% or less than or equal to about 1% or less than or equal to about 0.5% by weight of the dry weight of the powder composition. The total buffer salts may be present in an amount of greater than or equal to about 0.5% by weight of the dry weight of the composition. For example, in one embodiment the total buffer salts is present in an amount of from about 0.5% to about 7.5% or from about 0.5% to about 5.3% by weight of the dry weight of the powder composition.

In one embodiment of the invention, the composition further comprises an inhalable corticosteroid and/or a long-acting beta 2-agonist.

In one embodiment of the invention, the composition has a moisture content of less than or equal to about 5% by weight of the dry weight of the powder composition. For example, in one embodiment the moisture content is less than or equal to about 4% or less than or equal to about 3% or less than or equal to about 2% or less than or equal to about 1% by weight of the dry weight of the powder composition. For example, in one embodiment the composition has a moisture content of from about 1% to about 5% or from 2% to about 5% or from 3% to about 5% by weight of the dry weight of the powder composition.

In one embodiment of the invention, the composition has a moisture content of from about 2% to about 4% by weight of the dry weight of the powder composition after 1 m (month) or 2 m or 3 m or 6 m of storage at either 25° C./60% RH or 30° C./65% RH or 40° C./75% RH.

In one embodiment of the invention, the composition has a moisture content of from about 2% to about 5% by weight of the dry weight of the powder composition after 12 m of storage at either 25° C./60% RH or 30° C./65% RH.

In one embodiment of the invention, the composition has a moisture content of from about 2% to about 5% by weight of the dry weight of the powder composition after 24 m of storage at 30° C./65% RH.

In one embodiment of the invention, the composition has a glass transition temperature (T_{g}) equal to or greater than 60° C. or 65° C. The composition may have a T_{g} equal to or less than about 95° C., about 90° C., about 85° C., about 80° C. or about 75° C. For example, in one embodiment the composition has a T_{g} of from about 60° C. to about 95° C., from 65° C. to about 90° C., from 65° C. to about 85° C., from 65° C. to about 80° C. or from 65° C. to about 75° C.

In one embodiment of the invention, the composition has a T_{g} of from about 60° C. to about 95° C. or 65° to about 90° C. after 1 m or 2 m or 3 m or 6 m of storage at either 25° C./60% RH or 30° C./65% RH or 40° C./75% RH.

In one embodiment of the invention, the composition has a T_{g} of from about 65° C. to about 95° C. or from about 60° C. to about 90° C. after 12 m of storage at either 25° C./60% RH or 30° C./65% RH or 40° C./75% RH.

In one embodiment of the invention, the composition has a T_{g} of from about 60° C. to about 95° C. or from about 65° C. to about 90° C. after 24 m of storage at 30° C./65% RH.

In one embodiment of the invention, the composition has a particle size distribution (PSD) of d₁₀ less than or equal to about 10 μm. For example, in one embodiment the composition has a PSD of d₁₀ of less than or equal to about Sum or less than or equal to about 3 μm or less than or equal to about 2.5 μm or less than or equal to about 2 μm or less than or equal to about 1.5 μm. For example, in one embodiment the composition has a PSD of d₁₀ of from about 1 μm to about 3 μm or from 1 μm to about 2 μm.

In one embodiment, the composition has a PSD of d₁₀ that remains less than 2 μm or from 1 μm to about 2 μm after 1 m or 2 m or 3 m or 6 m or 12 m of storage at either 25° C./60% RH or 30° C./65% RH or 40° C./75% RH.

In one embodiment, the composition has a PSD of d₁₀ that remains less than 2 μm or from 1 μm to about 2 μm after 24 m of storage at 30° C./65% RH.

In one embodiment of the invention, the composition has a particle size distribution (PSD) of d₅₀ less than or equal to about 10 μm. For example, in one embodiment the composition has a PSD of d₅₀ of less than or equal to about 5 μm or less than or equal to about 4.5 μm or less than or equal to about 4 μm or less than or equal to about 3.5 μm or less than or equal to about 3 μm or less than or equal to about 2.5 μm. For example, in one embodiment the composition has a PSD of d₅₀ of from about 2 μm to about 5 μm or from about 2 μm to about 4 μm or from about 2 μm to about 3 μm.

In one embodiment, the composition has a PSD of d₅₀ that remains less than 4 μm or from 2 μm to about 4 μm after 1 m or 2 m or 3 m or 6 m or 12 m of storage at either 25° C./60% RH or 30° C./65% RH or 40° C./75% RH.

In one embodiment, the composition has a PSD of d₅₀ that remains less than 4 μm or from 2 μm to about 4 μm after 24 m of storage at 30° C./65% RH.

In one embodiment of the invention, the composition has a particle size distribution (PSD) of d₉₀ less than or equal to about 10 μm. For example, in one embodiment the composition has a PSD of d₉₀ of less than or equal to about 9.5 μm or less than or equal to about 9 μm or less than or equal to about 8.5 μm or less than or equal to about 8 μm or less than or equal to about 7.5 μm. For example, in one embodiment the composition has a PSD of d₉₀ of from about 3 μm to about 8 μm or from about 4 μm to about 8 μm or from about 4 μm to about 7 μm.

In one embodiment, the composition has a PSD of d₉₀ that remains less than 8 μm or from 4 μm to about 8 μm after 1 m or 2 m or 3 m or 6 m or 12 m of storage at either 25° C./60% RH or 30° C./65% RH or 40° C./75% RH.

In one embodiment, the composition has a PSD of d₉₀ that remains less than 8 μm or from 4 μm to about 8 μm after 24 m of storage at 30° C./65% RH.

In one embodiment of the invention, the particles of the composition have a particle size distribution (PSD) of d₁₀, less than or equal to about 3 μm. For example, in one embodiment the particles have a PSD of d₁₀ of less than or equal to about 2.5 μm or less than or equal to about 2 μm or less than or equal to about 1.5 μm. For example, in one embodiment the particles have a PSD of d₁₀ of from about 1 μm to about 3 μm or from 1 μm to about 2 μm.

In one embodiment of the invention, the particles of the composition have a particle size distribution (PSD) of d₅₀, less than or equal to about 5 μm. For example, in one embodiment the particles have a PSD of d₅₀ of less than or equal to about 4.5 μm or less than or equal to about 4 μm or less than or equal to about 3.5 μm or less than or equal to about 3 μm or less than or equal to about 2.5 μm. For example, in one embodiment the particles have a PSD of d₅₀ of from about 2 μm to about 5 μm or from about 2 μm to about 4 μm or from about 2 μm to about 3 μm.

In one embodiment of the invention, the particles of the composition have a particle size distribution (PSD) of d₉₀, less than or equal to about 10 μm. For example, in one embodiment the particles have a PSD of d₉₀ of less than or equal to about 9.5 μm or less than or equal to about 9 μm or less than or equal to about 8.5 μm or less than or equal to about 8 μm or less than or equal to about 7.5 μm. For example, in one embodiment the particles have a PSD of d₉₀ of from about 3 μm to about 8 μm or from about 4 μm to about 8 μm or from about 4 μm to about 7 μm.

In one embodiment the particles are spray-dried particles comprising a) an antagonistic antibody which binds IL-13, b) leucine and c) trehalose.

The leucine may be predominately present on the surface of the spray dried particles. Without wishing to be bound by theory, this may arise due to the leucine's hydrophobic and surface active properties.

In one embodiment of the invention, the nominal dose of the antibody is less than or equal to 25 mg, for example less than or equal to 20 mg, or less than or equal to 15 mg, or less than or equal to 10 mg, or less than or equal to 6 mg or less than or equal to 5 mg or less than or equal to 1 mg or less than or equal to 0.5 mg. The nominal dose of the antibody may be greater than or equal to about 0.5 mg, about 5 mg or about 10 mg. For example, in one embodiment the nominal dose of the antibody is from at least about 0.5 mg to about 20 mg, or from at least about 10 mg to about 20 mg.

In one embodiment of the invention, the delivered dose of the antibody is less than or equal to 15 mg, for example less than or equal to 14.8 mg, or less than or equal to 10 mg, or less than or equal to 7.4 mg, or less than or equal to 5 mg or less than or equal to 3.7 mg or less than or equal to 0.6 mg or less than or equal to 0.3 mg. The delivered dose of the antibody may be greater than or equal to about 0.3 mg. For example, in one embodiment the delivered dose of the antibody is from at least about 0.3 mg to about 14.8 mg.

In one embodiment of the invention, the respirable dose of the antibody is less than or equal to 8 mg, for example less than or equal to 7.2 mg, or less than or equal to 5 mg, or less than or equal to 3.6 mg or less than or equal to 2 mg or less than or equal to 1.8 mg, or less than or equal to 0.4 mg or less than or equal to 0.2 mg. The respirable dose of the antibody may be greater than or equal to about 0.2 mg. For example, in one embodiment the respirable dose of the antibody is from at least about 0.2 mg to about 7.2 mg.

In one embodiment of the invention, the composition used for treating asthma via inhalation comprises a nominal dose of antibody of less than or equal to 20 mg, such as less than or equal to 10 mg or less than or equal to 5 mg, or less than or equal to 1 mg or less than or equal to 0.5 mg. In particular embodiments, this nominal dose produces a delivered dose of 14.8 mg, 7.4 mg, 3.7 mg, 0.6 mg or 0.3 mg, respectively. In other embodiments of the invention the nominal dose of the composition is from at least about 0.5 mg and up to about 20 mg and provides a delivered dose of at least about 0.3 mg and up to about 14.8 mg.

In another embodiment of the invention, the composition used for treating asthma via inhalation comprises a nominal dose of antibody of less than or equal to 20 mg, such as less than or equal to 10 mg or less than or equal to 5 mg, or less than or equal to 1 mg or less than or equal to 0.5 mg. In particular embodiments, this nominal dose produces a respirable dose of 7.2 mg, 3.6 mg, 1.8 mg, 0.4 mg or 0.2 mg, respectively. In another embodiment of the invention the nominal dose of the composition is from at least about 0.5 mg and up to about 20 mg and provides a respirable dose of at least about 0.2 mg and up to about 7.2 mg.

In another embodiment, the composition provides a daily dose, which is the dose administered over a period of 24 hours. The daily dose may be received as a single dose or may be divided into a number of doses, for example given twice or three times daily. The daily dose may refer to the nominal dose, delivered dose or respirable dose.

In one embodiment, the powder of the invention has a tap density of less than or equal to about 0.7 g/cm³, for example less than or equal to about 0.62 g/cm³ or less than or equal to about 0.61 g/cm³, or less than or equal to about 0.60 g/cm³ or less than or equal to about 0.59 g/cm³ or less than or equal to about 0.58 g/cm³, or less than or equal to about 0.57 g/cm³. For example, in one embodiment, the powder of the invention has a tap density of from about 0.4 g/cm³ to about 0.7 g/cm³, or from about 0.55 g/cm³ to about 0.65 g/cm³.

Tap density can be measured by using instruments known to those skilled in the art such as, but not limited to, the Dual Platform Microprocessor Controlled Tap Density Tester (Vankel Technology, Cary, N.C.) or a GeoPyc™ instrument (Micrometrics Instrument Corp., Norcross, Ga. 30093). Tap density can be determined using the method of USP Bulk Density and Tapped Density, United States Pharmacopoeia convention, Rockville, Md. 39^{th} Supplement, Chapter 616, page 456, 2016. Preferably, the tap density is measured using a Copley Tap Density Volumeter (JV 2000).

For instance, the tap density was measured after 500 taps using a Copley Tap Density Volumeter (JV 2000).

It has surprisingly been found that subjects suffering from mild asthma having a FeNO level greater than 35 ppb at study entry demonstrated an FeNO reduction from about 13% to about 65% (from baseline) after 10 days of treatment with the compositions of the present invention. This was true for all three active treatment groups examined, i.e. the 0.5 mg, 10 mg and 20 mg nominal doses.

In one embodiment of the invention, the nominal dose of the antibody is from about 0.5 mg to about 20 mg and provides from about a 3% reduction to about a 25% reduction or at least about a 11.1% mean reduction in FeNO levels from baseline in a subject after 1 day of administering the nominal dose daily, or after administering a single treatment dose.

In one embodiment of the invention, the nominal dose of the antibody is from about 0.5 mg to about 20 mg and provides up to about a 32% reduction or at least about a 19.6% mean reduction in FeNO levels from baseline in a subject after 2 days of administering the nominal dose daily, or after administering 2 treatment doses.

In one embodiment of the invention, the nominal dose of the antibody is from about 0.5 mg to about 20 mg and provides from about a 13% reduction to about a 42% reduction or at least about a 33.5% mean reduction in FeNO levels from baseline in a subject after 3 days of administering the nominal dose daily, or after administering 3 treatment doses.

In one embodiment of the invention, the nominal dose of the antibody is from about 0.5 mg to about 20 mg and provides from about a 13% reduction to about a 65% reduction or at least about a 44.2% mean reduction in FeNO levels from baseline in a subject after 10 days of administering the nominal dose daily, or after administering 10 treatment doses.

In another embodiment of the invention the nominal dose of the antibody is from about 10 mg to about 20 mg and provides from about a 12% reduction to about a 38% reduction or at least about a 22.1% mean reduction in FeNO levels from baseline in a subject after 1 day of administering the nominal dose daily, or after administering a single treatment dose.

In one embodiment of the invention, the nominal dose of the antibody is from about 10 mg to about 20 mg and provides up to about a 49% reduction or at least about a 31% mean reduction in FeNO levels from baseline in a subject after 2 days of administering the nominal dose daily, or after administering 2 treatment doses.

In one embodiment of the invention, the nominal dose of the antibody is from about 10 mg to about 20 mg and provides from about a 13% reduction to about a 55% reduction or at least about a 42.2% mean reduction in FeNO levels from baseline in a subject after 3 days of administering the nominal dose daily, or after administering 3 treatment doses.

In one embodiment of the invention, the nominal dose of the antibody is from about 10 mg to about 20 mg and provides from about a 13% reduction to about a 75% reduction or at least about a 51.6% mean reduction in FeNO levels from baseline in a subject after 10 days of administering the nominal dose daily, or after administering 10 treatment doses.

In another embodiment of the invention, the nominal dose of the antibody is about 20 mg and provides from about a 22% reduction to about a 45% reduction or at least about a 21.7% mean reduction in FeNO levels from baseline in a subject after 1 day of administering the nominal dose daily, or after administering a single treatment dose.

In one embodiment of the invention, the nominal dose of the antibody is about 20 mg and provides from about a 6% reduction to about a 59% reduction or at least about a 39% mean reduction in FeNO levels from baseline in a subject after 2 days of administering the nominal dose daily, or after administering 2 treatment doses.

In one embodiment of the invention, the nominal dose of the antibody is about 20 mg and provides from about a 13% reduction to about a 70% reduction or at least about a 46.3% mean reduction in FeNO levels from baseline in a subject after 3 days of administering the nominal dose daily, or after administering 3 treatment doses.

In one embodiment of the invention, the nominal dose of the antibody is about 20 mg and provides from about a 13% reduction to about a 84% reduction or about a 54.2% mean reduction in FeNO levels from baseline in a subject after 10 days of administering the nominal dose daily, or after administering 10 treatment doses.

In studies that have been carried out, the rapid, durable, and dose-related reduction of FeNO versus placebo that was observed in the mild asthmatic subjects was also maintained for at least 4 days following the last administered treatment dose. Statistically significant (p<0.05) changes were reported for treatment groups subjected to 10 mg and 20 mg daily nominal doses.

In one embodiment of the invention, the nominal dose of the antibody is from about 0.5 mg to about 20 mg and maintains from about a 13% reduction to about a 65% reduction or at least about a 44.2% mean reduction in FeNO levels from baseline in a subject 1 day after the last administered treatment dose.

In one embodiment of the invention, the nominal dose of the antibody is from about 0.5 mg to about 20 mg and maintains from about a 6% reduction to about a 64% reduction or at least about a 44.3% mean reduction in FeNO levels from baseline in a subject 2 days after the last administered treatment dose.

In one embodiment of the invention, the nominal dose of the antibody is from about 0.5 mg to about 20 mg and maintains up to about a 65% reduction or at least about a 41.7% mean reduction in FeNO levels from baseline in a subject 3 days after the last administered treatment dose.

In one embodiment of the invention, the nominal dose of the antibody is from about 0.5 mg to about 20 mg and maintains from about a 2% reduction to about a 61% reduction or at least about a 39% mean reduction in FeNO levels from baseline in a subject 4 days after the last administered treatment dose.

In one embodiment of the invention, the nominal dose of the antibody is from about 10 mg to about 20 mg and maintains from about a 13% reduction to about a 75% reduction or at least about a 51.6% mean reduction in FeNO levels from baseline in a subject 1 day after the last administered treatment dose.

In one embodiment of the invention, the nominal dose of the antibody is from about 10 mg to about 20 mg and maintains from about a 6% reduction to about a 64% reduction or at least about a 47.9% mean reduction in FeNO levels from baseline in a subject 2 days after the last administered treatment dose.

In one embodiment of the invention, the nominal dose of the antibody is from about 10 mg to about 20 mg and maintains up to about a 66% reduction or at least about a 46.1% mean reduction in FeNO levels from baseline in a subject 3 days after the last administered treatment dose.

In one embodiment of the invention, the nominal dose of the antibody is from about 10 mg to about 20 mg and maintains from about a 12% reduction to about a 61% reduction or at least about a 41.5% mean reduction in FeNO levels from baseline in a subject 4 days after the last administered treatment dose.

In one embodiment of the invention, the nominal dose of the antibody is from about 20 mg and maintains from about a 13% reduction to about a 84% reduction or at least about a 54.2% mean reduction in FeNO levels from baseline in a subject 1 day after the last administered treatment dose.

In one embodiment of the invention, the nominal dose of the antibody is from about 20 mg and maintains from about a 6% reduction to about a 79% reduction or at least about a 53% mean reduction in FeNO levels from baseline in a subject 2 days after the last administered treatment dose.

In one embodiment of the invention, the nominal dose of the antibody is from about 20 mg and maintains up to about a 83% reduction or at least about a 51% mean reduction in FeNO levels from baseline in a subject 3 days after the last administered treatment dose.

In one embodiment of the invention, the nominal dose of the antibody is from about 20 mg and maintains up to about a 77% reduction or about a 41.5% mean reduction in FeNO levels from baseline in a subject 4 days after the last administered treatment dose.

In one embodiment of the invention, the nominal dose of the antibody is from about 0.5 mg and provides improvements of up to about 0.43 L or a mean improvement of about 0.2 L in FEV₁ from baseline in a subject after 2 hours of administering the nominal dose. For example, in one embodiment a nominal dose of from about 0.5 mg provides improvements of more than 0.15 L in FEV₁ from baseline in a subject after 2 hours of administering the nominal dose.

In one embodiment of the invention, the nominal dose of the antibody is from about 0.5 mg and provides improvements of up to about 0.43 L or a mean improvement of about 0.2 L in FEV₁ from baseline in a subject after 2 hours of administering 1 treatment dose. For example, in one embodiment a nominal dose of from about 0.5 mg provides improvements of more than 0.15 L in FEV₁ from baseline in a subject after 2 hours of administering 1 treatment dose.

In one embodiment of the invention, the nominal dose of the antibody is from about 10 mg and provides improvements of up to about 0.58 L or a mean improvement of about 0.13 L in FEV₁ from baseline in a subject after 2 hours of administering the nominal dose.

For example, in one embodiment a nominal dose of from about 10 mg provides improvements of more than 0.10 L in FEV₁ from baseline in a subject after 2 hours of administering the nominal dose.

In one embodiment of the invention, the nominal dose of the antibody is from about 10 mg and provides improvements of up to about 0.58 L or a mean improvement of about 0.13 L in FEV₁ from baseline in a subject after 2 hours of administering 1 treatment dose. For example, in one embodiment a nominal dose of from about 10 mg provides improvements of more than 0.10 L in FEV₁ from baseline in a subject after 2 hours of administering 1 treatment dose.

In one embodiment of the invention, the nominal dose of the antibody is from about 20 mg and provides improvements of up to about 0.57 L or a mean improvement of about 0.18 L in FEV₁ from baseline in a subject after 2 hours of administering the nominal dose. For example, in one embodiment a nominal dose of from about 20 mg provides improvements of more than 0.15 L in FEV₁ from baseline in a subject after 2 hours of administering the nominal dose.

In one embodiment of the invention, the nominal dose of the antibody is from about 20 mg and provides improvements of up to about 0.57 L or a mean improvement of about 0.18 L in FEV₁ from baseline in a subject after 2 hours of administering 1 treatment dose.

For example, in one embodiment a nominal dose of from about 20 mg provides improvements of more than 0.15 L in FEV₁ from baseline in a subject after 2 hours of administering 1 treatment dose.

In one embodiment of the invention, the nominal dose of the antibody is about 0.5 mg and provides improvements of up to about 0.61 L or a mean improvement of about 0.26 L in FEV₁ from baseline in a subject after 10 days of administering the nominal dose daily. For example, in one embodiment a nominal dose of from about 0.5 mg provides improvements of more than 0.15 L or more than 0.2 L or more than 0.25 L in FEV₁ from baseline in a subject after 10 days of administering the nominal dose daily.

In one embodiment of the invention, the nominal dose of the antibody is about 0.5 mg and provides improvements of up to about 0.61 L or a mean improvement of about 0.26 L in FEV₁ from baseline in a subject after administering 10 treatment doses. For example, in one embodiment a nominal dose of from about 0.5 mg provides improvements of more than 0.15 L or more than 0.2 L or more than 0.25 L in FEV₁ from baseline in a subject after administering 10 treatment doses.

In one embodiment of the invention, the nominal dose of the antibody is about 0.5 mg and maintains improvements of up to about 0.69 L or a mean improvement of about 0.32 L in FEV₁ from baseline in a subject 4 days after the last administered treatment dose. For example, in one embodiment a nominal dose of from about 0.5 mg maintains improvements of more than 0.15 L or more than 0.2 L or more than 0.25 L or more than 0.30 L in FEV₁ from baseline in a subject 4 days after the last administered treatment dose.

In one embodiment of the invention, the nominal dose of the antibody is about 20 mg and provides improvements of up to about 0.48 L or a mean improvement of about 0.19 L in FEV₁ from baseline in a subject after 10 days of administering the nominal dose daily. For example, in one embodiment a nominal dose of from about 20 mg provides improvements of more than 0.10 L or more than 0.15 L in FEV₁ from baseline in a subject after 10 days of administering the nominal dose daily.

In one embodiment of the invention, the nominal dose of the antibody is about 20 mg and provides improvements of up to about 0.48 L or a mean improvement of about 0.19 L in FEV₁ from baseline in a subject after administering 10 treatment doses. For example, in one embodiment a nominal dose of from about 20 mg provides improvements of more than 0.10 L or more than 0.15 L in FEV₁ from baseline in a subject after administering 10 treatment doses.

In one embodiment of the invention, the nominal dose of the antibody is about 20 mg and maintains improvements of up to about 0.64 L or a mean improvement of about 0.27 L in FEV₁ from baseline in a subject for 4 days after the last administered treatment dose. For example, in one embodiment a nominal dose of from about 20 mg maintains improvements of more than 0.10 L or more than 0.15 L or more then 0.20 L or more than 0.25 L in FEV₁ from baseline in a subject for 4 days after the last administered treatment dose.

In one aspect of the invention, there is provided a process for preparing an inhalable dry powder composition, the process comprising the steps of;
(i) preparing a first aqueous solution and/or suspension comprising leucine and trehalose;
(ii) preparing a second aqueous solution and/or suspension comprising antagonistic antibody which binds human IL-13 and buffer salt;
(iii) mixing the first and second aqueous solutions and/or suspensions from steps (i) and (ii) to form a feedstock solution and/or suspension; and
(iv) spray-drying the feedstock solution and/or suspension from step (iii).

In one embodiment of the invention, the antibody from step (ii) is selected from the group consisting of: a complete antibody molecule having full length heavy and light chains or a fragment thereof, such as a Fab, modified Fab′, Fab′, F(ab′)₂, Fv, VH, VL or scFv fragment.

In a further embodiment of the invention, the antibody used in step (ii) comprises a heavy chain, wherein the variable domain of the heavy chain comprises the sequence given in SEQ ID NO:3 and, additionally comprises a light chain, wherein the variable domain of the light chain comprises the sequence given in SEQ ID NO:1.

In an embodiment of the invention, the antibody used in step (ii) is CDP7766.

In some embodiments of the invention, the antibody comprises a light chain that has at least 60% homology, identity or similarity to the sequence given in SEQ ID NO:1, or at least 70%, at least 80%, at least 90%, at least 95% or at least 98% homology, identity or similarity.

In some embodiments of the invention, the antibody comprises a heavy chain that has at least 60% homology, identity or similarity to the sequence given in SEQ ID NO:3, or at least 70%, at least 80%, at least 90%, at least 95% or at least 98% homology, identity or similarity.

In one embodiment the first aqueous solution and/or suspension from step (i) is added to the second aqueous solution and/or suspension from step (ii) and the combined solutions and/or solutions are then mixed to form the feedstock solution and/or suspension.

In another embodiment the second aqueous solution and/or suspension from step (ii) is added to the first aqueous solution and/or suspension from step (i) and the combined solutions and/or solutions are then mixed to form the feedstock solution and/or suspension.

In one embodiment the buffer salt used in step (ii) is phosphate buffered saline (PBS). PBS is frequently used in biological research due to its isotonic nature and non-toxicity to cells. PBS buffer components include sodium chloride (NaCl) and phosphate salt. PBS generally contains between 125 mM and 138 mM NaCl and between 2 mM and 10 mM phosphate salt such as sodium phosphate (Na₂HPO₄).

In a further embodiment of the invention, the NaCl concentration is less than about 125 mM, for example less than or equal to about 100 mM, such as less than or equal to about 50 mM, in one embodiment less than or equal to about 25 mM. The NaCl concentration may be greater than or equal to about 10 mM or about 25 mM. For example, in one embodiment, the PBS NaCL concentration is from about 10 mM to about 25 mM, or from about 15 mM to about 20 mM.

In another embodiment of the invention the phosphate salt component is selected from the group consisting of: sodium phosphate (Na₂HPO₄); potassium phosphate (KH₂PO₄); sodium pyrophosphate dibasic, sodium triphosphate and/or sodium polyphosphate. In a preferred embodiment the phosphate salt in the feedstock is Na₂HPO₄ and/or KH₂PO_{4.}

In one embodiment of the present invention, the Na₂HPO₄ concentration is less than or equal to about 20 mM, for example less than or equal to about 15 mM, such as less than or equal to about 10 mM. The Na₂HPO₄ concentration may ne greater than or equal to about 5 mM or about 8 mM. For example, in one embodiment, the PBS Na₂HPO₄ concentration is from about 5 mM to about 15 mM, or from about 8 mM to about 12 mM.

In a further embodiment of the present invention, the PBS buffer salt used in step (ii) comprises NaCl and Na₂HPO₄, wherein the NaCl concentration is less than 125 mM and the Na₂HPO₄ concentration is less than 20 mM. In one embodiment the NaCl concentration is from about 10 mM to about 25 mM and the Na₂HPO₄ concentration is from about 5 mM to about 15 mM.

In one embodiment of the invention the pH of the feedstock solution and/or suspension is less than or equal to about pH 7, for example less than or equal to about pH 6.5, or less than or equal to about pH 6. The pH of the feedstock solution and/or suspension may be greater than or equal to about pH 6. For example, in one embodiment, the pH of the feedstock solution and/or suspension is from about pH 7 to about pH 6.

In another embodiment of the invention the pH of the feedstock solution and/or suspension is adjusted with hydrochloric acid.

In one embodiment of the invention the total solids content of the feedstock solution and/or suspension is less than or equal to about 6% w/v, for example less than or equal to about 5% w/v, less than or equal to about 4.8% w/v, less than or equal to about 4% w/v, less than or equal to about 3.8% w/v or less than or equal to about 3% w/v. The total solids content of the feedstock solution and/or suspension may be greater than or equal to about 3% w/v or about 3.8% w/v. For example, in one embodiment, the total solids content of the feedstock solution/suspension is from about 3% w/v to about 6% w/v, or from about 3.8% w/v to about 4.8% w/v.

The process of the invention may be carried out on any suitable spray drying apparatus. A suitable spray drying apparatus is for example the Niro Mobile Minor spray dryer.

In one embodiment of the invention the inlet temperature of the spray drying apparatus is from about 115° C. to about 150° C., or from about 120° C. to about 145° C., or from about 120° C. to about 140° C. or from about 130° C. to about 145° C.

In another embodiment of the invention the outlet temperature of the spray drying apparatus is from about 45° C. to about 85° C., or from about 55° C. to about 75° C. More preferably the outlet temperature is less than or equal to about 65° C.

In one embodiment of the invention the feedstock solution and/or suspension comprising the antagonistic antibody, buffer salts, trehalose and leucine can include additional actives and/or excipients. For example, the feedstock solution may further comprise a corticosteroid and/or a long-acting beta 2-agonist.

In one aspect of the invention, there are provided inhalable particles comprising a) an antagonistic antibody which binds human IL-13, b) leucine and c) trehalose.

In one embodiment of the invention, the antibody is selected from the group consisting of: a complete antibody molecule having full length heavy and light chains or a fragment thereof, such as a Fab, modified Fab′, Fab′, F(ab′)₂, Fv, VH, VL or scFv fragment.

In a further embodiment of the invention, the antibody comprises a heavy chain, wherein the variable domain of the heavy chain comprises the sequence given in SEQ ID NO:3 and, additionally comprises a light chain, wherein the variable domain of the light chain comprises the sequence given in SEQ ID NO:1.

In an embodiment of the invention, the antibody is CDP7766.

In some embodiments of the invention, the antibody comprises a light chain that has at least 60% homology, identity or similarity to the sequence given in SEQ ID NO:1, or at least 70%, at least 80%, at least 90%, at least 95% or at least 98% homology, identity or similarity.

In some embodiments of the invention, the antibody comprises a heavy chain that has at least 60% homology, identity or similarity to the sequence given in SEQ ID NO:3, or at least 70%, at least 80%, at least 90%, at least 95% or at least 98% homology, identity or similarity.

In one embodiment of the invention the antibody, leucine and trehalose are co-spray dried.

In a further embodiment the particles are mixed with further active ingredient(s) and/or excipients. For example, the particles may be mixed with a corticosteroid and/or a long-acting beta 2-agonist.

In one aspect of the invention, there is provided a container comprising an inhalable powder composition comprising a) an antagonistic antibody which binds human IL-13, b) leucine and c) trehalose.

In one embodiment of the invention, the antibody in the container is selected from the group consisting of: a complete antibody molecule having full length heavy and light chains or a fragment thereof, such as a Fab, modified Fab′, Fab′, F(ab′)₂, Fv, VH, VL or scFv fragment.

In one embodiment of the invention, the antibody in the container comprises a heavy chain, wherein the variable domain of the heavy chain comprises the sequence given in SEQ ID NO:3 and, additionally comprises a light chain, wherein the variable domain of the light chain comprises the sequence given in SEQ ID NO:1.

In an embodiment of the invention, the antibody in the container is CDP7766.

In some embodiments of the invention, the antibody in the container comprises a light chain that has at least 60% homology, identity or similarity to the sequence given in SEQ ID NO:1, or at least 70%, at least 80%, at least 90%, at least 95% or at least 98% homology, identity or similarity.

In some embodiments of the invention, the antibody in the container comprises a heavy chain that has at least 60% homology, identity or similarity to the sequence given in SEQ ID NO:3, or at least 70%, at least 80%, at least 90%, at least 95% or at least 98% homology, identity or similarity.

Containers which are suitable for use in the present invention include a bulk storage container, such as a multi-dose reservoir for a dry powder inhaler, and unit dose containers such as a capsule or a blister. A capsule may be formed from various materials e.g. gelatine, cellulose derivatives such as hydroxypropyl methylcellulose (HPMC) or hydroxypropylcellulose (HPC), starch, starch derivatives, chitosan or synthetic plastics, while the blister may be provided in the form of a blister pack or blister strip.

In one embodiment the container is a blister such as a unit dose foil blister. For example, in one embodiment the unit dose foil blister consists of a base foil made from a polyamide/aluminium/polyvinylchloride (oPA/Al/PVC) foil laminate sealed with an aluminium lid foil.

In another embodiment the aluminium lid foil includes an over-lacquer. This enables direct printing, for example of batch information onto the foil lid.

In one embodiment the blister pockets are made by cold forming the base foil, which is then heat-sealed with the lid foil following blister filling.

In one embodiment of the invention, the composition or particles are suitable for filling directly into a container by hand, machine or by automated filling. For example in one embodiment the composition or particles are filled into a container such as a blister by hand or by using a fill to weight powder filling system.

In one embodiment a blister fill weight of from about 10 mg to about 30 mg was used, such as about 12.5 mg. For example, in one embodiment the blister fill weight is from about 10 mg to about 25 mg or from 10 mg to about 20 mg or from 11.5 mg to about 13.5 mg.

In one aspect of the invention, there is provided a dry powder inhaler comprising an inhalable powder composition comprising a) an antagonistic antibody which binds human IL-13, b) leucine and c) trehalose.

In one embodiment of the invention, the antibody administered by the dry powder inhaler is selected from the group consisting of: a complete antibody molecule having full length heavy and light chains or a fragment thereof, such as a Fab, modified Fab′, Fab′, F(ab′)₂, Fv, VH, VL or scFv fragment.

In one embodiment of the invention the antibody administered by the dry powder inhaler comprises a heavy chain, wherein the variable domain of the heavy chain comprises the sequence given in SEQ ID NO:3 and, additionally comprises a light chain, wherein the variable domain of the light chain comprises the sequence given in SEQ ID NO:1.

In an embodiment of the invention, the antibody administered by the dry powder inhaler is CDP7766.

In some embodiments of the invention, the antibody administered by the dry powder inhaler comprises a light chain that has at least 60% homology, identity or similarity to the sequence given in SEQ ID NO:1, or at least 70%, at least 80%, at least 90%, at least 95% or at least 98% homology, identity or similarity.

In some embodiments of the invention, the antibody administered by the dry powder inhaler comprises a heavy chain that has at least 60% homology, identity or similarity to the sequence given in SEQ ID NO:3, or at least 70%, at least 80%, at least 90%, at least 95% or at least 98% homology, identity or similarity.

In a dry powder inhaler, the dose to be administered is stored in the form of a non-pressurized dry powder and, on actuation of the inhaler the particles of the powder are expelled from the device in the form of a cloud of finely dispersed particles that may be inhaled by the patient.

Dry powder inhalers can be “passive” devices in which the patient's breath is the only source of gas which provides a motive force in the device. Examples of “passive” dry powder inhaler devices include the Rotahaler™ and Diskhaler™ (GlaxoSmithKline), the Monohaler™ (Miat), the GyroHaler™ unit dose inhaler as described in WO2010/086285 (Vectura), the Turbohaler™ (AstraZeneca) and Novolizer™ (Viatris GmbH).

Alternatively, “active” devices may be used, in which a source of compressed gas or alternative energy source is used. Examples of suitable active devices include Aspirair™ (Vectura) and the active inhaler device produced by Nektar Therapeutics (as covered by U.S. Pat. No. 6,257,233).

It is generally considered that compositions perform differently when dispensed using passive and active type inhalers. Passive devices create less turbulence within the device and the powder particles move more slowly when they leave the device. This leads to some of the metered dose remaining in the device and, depending on the nature of the composition, less deagglomeration upon actuation. However, when the slow-moving cloud is inhaled, less deposition in the throat is often observed. In contrast, active devices create more turbulence when they are activated. This results in more of the metered dose being extracted from the blister or capsule and better deagglomeration as the powder is subjected to greater shear forces. However, the particles leave the device faster than with passive devices and this can lead to an increase in throat deposition.

In one embodiment the dry powder composition of the present invention can be administered with a passive or active inhaler device (multi or unit dose devices). For example, in one embodiment the inhaler is a passive unit dose inhaler such as the unit dose device described in WO2010/086285.

In another aspect of the invention, there is provided an inhalable powder composition comprising a) an antagonistic antibody which binds human IL-13, b) leucine and c) trehalose, for use in the treatment of asthma.

Similarly, the invention provides the use of an inhalable powder composition comprising a) an antagonistic antibody which binds human IL-13, b) leucine and c) trehalose in the manufacture of a medicament for the treatment of asthma.

In a related aspect, the invention provides a method of treatment of asthma in a subject suffering from or susceptible to that condition, which method comprises the administration to the subject of an inhalable powder composition comprising a) an antagonistic antibody which binds human IL-13, b) leucine and c) trehalose.

In one embodiment of the invention, the antibody used is selected from the group consisting of: a complete antibody molecule having full length heavy and light chains or a fragment thereof, such as a Fab, modified Fab′, Fab′, F(ab′)₂, Fv, VH, VL or scFv fragment.

In one embodiment of the invention the antibody used comprises a heavy chain, wherein the variable domain of the heavy chain comprises the sequence given in SEQ ID NO:3 and, additionally comprises a light chain, wherein the variable domain of the light chain comprises the sequence given in SEQ ID NO:1.

In an embodiment of the invention, the antibody used is CDP7766.

In some embodiments of the invention, the antibody used comprises a light chain that has at least 60% homology, identity or similarity to the sequence given in SEQ ID NO:1, or at least 70%, at least 80%, at least 90%, at least 95% or at least 98% homology, identity or similarity.

In some embodiments of the invention, the antibody used comprises a heavy chain that has at least 60% homology, identity or similarity to the sequence given in SEQ ID NO:3, or at least 70%, at least 80%, at least 90%, at least 95% or at least 98% homology, identity or similarity.

In one embodiment of the invention, the asthma is mild asthma.

In one embodiment of the invention, the subjects are adults.

In one embodiment of the invention, the asthmatic subjects do not receive inhaled corticosteroids (i.e. ICS naïve subjects). In another embodiment of the invention, the asthmatic subjects additionally receive inhaled or oral corticosteroids.

The treatment may result in a reduced FeNO level of from about 3% to about 45% or from about 3% to about 25% or from about 12% to about 28% or from about 22% to about 45% or more than 10% or more than 20% or more than 30% or more than 40% (from baseline) after 1 day of treatment, or in a reduced FeNO level of up to 59% or up to 49% or up to 32% or from about 6% to about 59% or more than 20% or more than 30% or more than 40% or more than 50% (from baseline) after 2 days of treatment, or in a reduced FeNO level of from about 13% to about 70% or from about 13% to about 55% or from 13% to about 42% or more than 30% or more than 40% or more than 50% or more than 60% (from baseline) after 3 days of treatment, or in a reduced FeNO level of from about 13% to about 84% or from about 13% to about 75% or from 13% to about 65% or more than 40% or more than 50% or more than 60% or more than 70% (from baseline) after 10 days of treatment.

The treatment may result in improvements in FEV₁ of up to about 0.61 L or up to about 0.48 L or more than 0.1 L or more than 0.15 L (from baseline) after 10 days of treatment.

The treatment may result in a maintained reduction in FeNO levels of from about 13% to about 84% or from about 13% to about 75% or from about 13% to about 65% or more than 10% or more than 20% or more than 30% or more than 40% (from baseline) in a subject 1 day after the last administered treatment dose, or in a maintained reduction in FeNO levels of from about 6% to about 79% or from about 6% to about 64% or more than 20% or more than 30% or more than 40% or more than 50% or more than 60% (from baseline) in a subject 2 days after the last administered treatment dose, or in a maintained reduction in FeNO levels of up to 83% or up to 66% or up to 65% or more than 40% or more than 50% or more than 60% or more than 70% (from baseline) in a subject 3 days after the last administered treatment dose, or in a maintained reduction in FeNO levels of from about 2% to about 77% or from about 2% to about 61% or from about 12% to about 61% or more than 40% or more than 50% or more than 60% or more than 70% (from baseline) in a subject 4 days after the last administered treatment dose.

The treatment may result in maintained improvements in FEV₁ of up to about 0.69 L or up to about 0.64 L or more than 0.15 L or more than 0.20 L or more than 0.25 L (from baseline) in a subject 4 days after the last administered treatment dose.

In one embodiment of the invention the treatment dose refers to the nominal dose, the delivered dose or the respirable dose.

In another aspect of the invention, there is provided a pharmaceutical kit comprising:
(i) an inhalable powder composition comprising a) an antagonistic antibody which binds human IL-13, b) leucine and c) trehalose, and
(ii) a dry powder inhaler.

In one embodiment of the invention, the antibody in the pharmaceutical kit composition is selected from the group consisting of: a complete antibody molecule having full length heavy and light chains or a fragment thereof, such as a Fab, modified Fab′, Fab′, F(ab′)₂, Fv, VH, VL or scFv fragment.

In a further embodiment of the invention, the antibody in the pharmaceutical kit composition comprises a heavy chain, wherein the variable domain of the heavy chain comprises the sequence given in SEQ ID NO:3 and, additionally comprises a light chain, wherein the variable domain of the light chain comprises the sequence given in SEQ ID NO:1.

In an embodiment of the invention, the antibody in the pharmaceutical kit composition is CDP7766.

In some embodiments of the invention, the antibody in the pharmaceutical kit composition comprises a light chain that has at least 60% homology, identity or similarity to the sequence given in SEQ ID NO:1, or at least 70%, at least 80%, at least 90%, at least 95% or at least 98% homology, identity or similarity.

In some embodiments of the invention, the antibody in the pharmaceutical kit composition comprises a heavy chain that has at least 60% homology, identity or similarity to the sequence given in SEQ ID NO:3, or at least 70%, at least 80%, at least 90%, at least 95% or at least 98% homology, identity or similarity.

In one embodiment of the invention the composition in the pharmaceutical kit is preferably provided in sterile containers such as blisters, each holding the appropriate nominal dose required to administer an effective delivered dose or respirable dose. For example, the nominal dose of the composition is from at least about 0.5 mg and up to about 20 mg and provides a delivered dose of at least about 0.3 mg and up to about 14.8 mg or the nominal dose of the composition is from at least about 0.5 mg and up to about 20 mg and provides a respirable dose of at least about 0.2 mg and up to about 7.2 mg.

In a further embodiment the inhaler is any type of dry powder inhaler suitable for administering the composition. Preferably the inhaler in the pharmaceutical kit is a passive dry powder inhaler, such as the unit dose inhaler described in WO2010/086285 or a multi-unit dose inhaler.

### The VR942/1/001 Clinical Study

This study investigated the safety, tolerability, pharmacodynamics (PD) and pharmacokinetics (PK) profile of VR942 (i.e. the drug product containing the active CDP7766) after single ascending doses in healthy subjects, and repeated ascending doses (once daily for 10 days) in mild asthmatics (as defined by the GINA guidelines). The doses selected in this study were chosen based on the NOAEL and the A. suum challenge model of asthma in cynomolgus monkeys. The study was designed to provide sufficient confidence in the safety profile of VR942, and to explore its PD effect in mild asthmatics, to allow progression to further studies. The primary variables of vital signs, ECG, physical examination, laboratory safety tests, spirometry, DLCO, AEs, adverse device effect and adverse events were evaluated in the assessment of safety and tolerability.

### Part 1

Part 1 was a randomised, double-blind, placebo-controlled, single ascending-dose (SAD) study in 40 healthy subjects. Subjects were allocated to one of five groups of eight subjects. Each subject received a single inhaled dose of VR942, or matching placebo. Doses administered are shown in Table 1.

TABLE 1
the VR942 doses used in Part 1 of the study.

| | Number | Delivered Dose | Nominal | | Respirable |
|---|---|---|---|---|---|
| | of | (Nominal Dose) | Dose per | Number of | Dose (FPM) |
| Group | Subjects | (mg) | Blister (mg) | Blisters | (mg) |
| 1 | 8 | 0.3 (0.5) | 0.5 | 1 | 0.2 |
| 2 | 8 | 0.6 (1) | | 2 | 0.4 |
| 3 | 8 | 3.7 (5) | 5.0 | 1 | 1.8 |
| 4 | 8 | 7.4 (10) | | 2 | 3.6 |
| 5 | 8 | 14.8 (20) | | 4 | 7.2 |

There was at least 7 days between dosing of each group. In each group, six subjects took VR942 and two subjects took matching placebo (3:1 ratio of VR942: placebo). A sentinel dosing approach was used at each new ascending dose level. In each group, two sentinel subjects were dosed at least 47 hours before dosing the remaining subjects. Provided the investigator had no safety concerns, the remaining subjects in that group were dosed, at intervals of at least 10 minutes. To maintain the blinded nature of the study, the sentinel pair of subjects were dosed in a 1:1 ratio of VR942:placebo.

Each blister administered to sentinel pair subjects during Part 1 was inspected by a blinded representative of the study sponsor. Where the inspection suggested that the blister contents had not evacuated as expected, then the investigator, in consultation with the sponsor, retained the option to replace the sentinel subjects—i.e. to assess another sentinel pair before dosing of remaining six subjects in the group. If that happened, the original sentinel subjects were to complete all assessments, with the exception of PK blood sampling.

Subjects were screened within 28 days of their first dose of trial medication. They were resident on the ward from the day before dosing (Day −1) until completion of procedures at 72 hours after their dose of trial medication (Day 4), and returned to the ward for an outpatient visit on Day 14 (±2 days).

All subjects attended a final follow-up visit 28 days (±2 days) after their dose of trial medication.

### Part 2

Part 2 was a randomised, double-blind, placebo-controlled, repeated ascending-dose study in mild asthmatics (N=52 were not using ICS, with N=1 using a low dose ICS).

Subjects were allocated to one of three groups. Each subject received once-daily doses of VR942, or matching placebo, for 10 days. Planned doses, group sizes, and the ratios of subjects allocated to receive VR942 and placebo, are shown in Table 2.

TABLE 2
the group sizes and ratio of VR942:placebo used in Part 2 of the study.

| | Delivered Dose | | | Respirable |
|---|---|---|---|---|
| | (Nominal Dose) | Number of | Ratio of VR942: | Dose (FPM) |
| Group | (mg) | subjects | placebo | (mg) |
| 1 | 0.3 (0.5) | 9 | 2:1 | 0.2 |
| 2 | 7.4 (10) | 9 | 2:1 | 3.6 |
| 3 | 14.8 (20) | 35 | 3:2 | 7.2 |

For each ascending dose level, there was at least 7 days between the final dose of the previous group and the first dose of the next group. Subjects within each group were dosed at intervals of at least 10 minutes.

Subjects were screened within 28 days of their first dose of trial medication; or within 14 to 28 days before their first dose for subjects in Part 2 who were taking ICS. They were resident on the ward from the day before dosing (Day −1) until completion of procedures at 96 hours following their final dose of trial medication (Day 14). They also attended a follow-up visit 28 days (±2 days) following their final dose of trial medication.

### EXAMPLES

The following examples are provided to illustrate the invention but should not be construed as limiting the invention.

### Example 1—Spray-Dried Formulations

The dry powder compositions (Table 3) were prepared by spray drying the aqueous feedstock solutions and/or suspensions using a Niro Pharma SD Spray Dryer equipped with a two fluid nozzle, under the following conditions:

| | |
|---|---|
| Feed Rate | 1.1 kg/hr⁻¹ |
| Drying Air Flow | 85 kg/hr⁻¹ |
| Atomisation Flow | 8.0 kg/hr⁻¹ |
| Outlet Temperature | 65° C. |

TABLE 3
summarises the VR942 compositions formulated. Contents are
dry contents as % w/w.

| | CDP7766 Drug | | | |
|---|---|---|---|---|
| | Substance (% | Buffer Salts | Leucine | Trehalose |
| Product | w/w) | (% w/w) | (% w/w) | (% w/w) |
| Placebo | 0.0 | 0.0 | 10 | 90.0 |
| VR942 0.5 mg | 4.0 | 0.5 | 10 | 85.5 |
| VR942 5.0 mg | 40.0 | 5.1 | 10 | 44.9 |

The bulk spray-dried VR942 samples were collected into glass jars at <11% RH, and sealed with parafilm and stored in a desiccator. Samples were then used to fill unit dose blisters under low humidity (<11% RH) to a fill weight of between 11.5 and 13.5 mg, for example providing a 12.5 mg fill weight. Blisters were stored under various conditions (25° C./60% RH, 30° C./65% RH and 40° C./75% RH) and subsequently analysed at various time points (up to 24 months). Analysis included determining mean particle size distribution (PSD); mean glass transition (T_{g}) by DSC; mean moisture content (KF) and mean potency by IL-13 binding activity (ELISA) compared against reference standard.

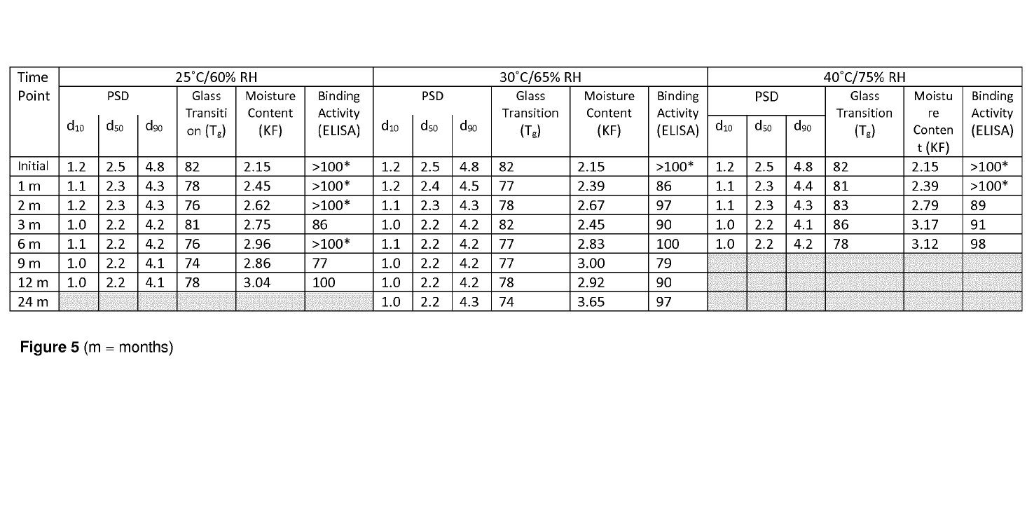

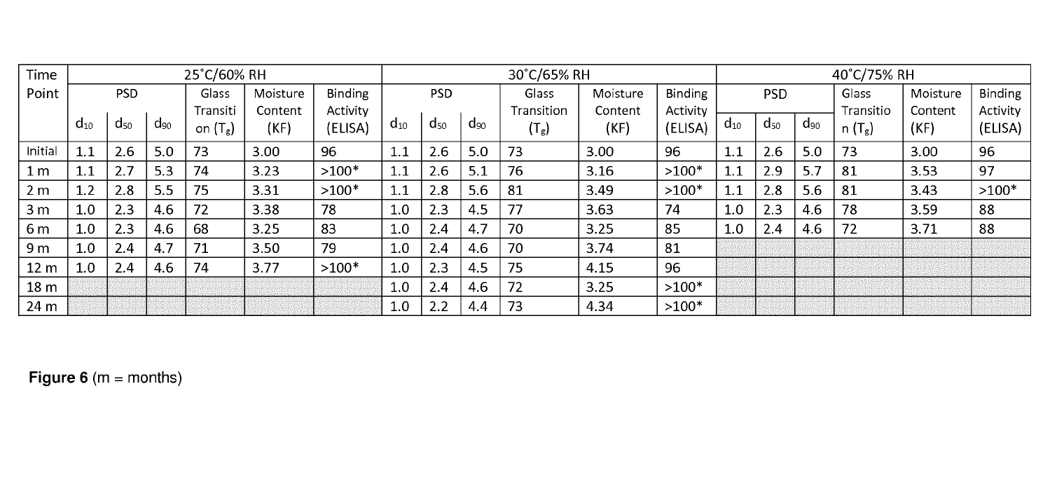

The particle size distribution (PSD) of the spray dried powder formulations were determined using a Malvern Mastersizer 3000 laser diffractometer and wet dispersion unit (Hydro MV). Particle size distribution for each sample was measured six times. The results are shown in FIG. 5 (for 0.5 mg doses) and FIG. 6 (for 5 mg doses).

DSC is a thermo-analytical technique in which the difference in the amount of heat required to increase the temperature of a sample, and reference is measured as a function of temperature. This essentially evaluates the changes in glass transition temperature (T_{g}). The results are shown in FIG. 5 (for 0.5 mg doses) and FIG. 6 (for 5 mg doses).

Karl Fisher analysis was used to assess the % moisture content of formulations. A titration vessel reagent of Hydranal® Coulomat: AG Oven was used. The spray dried formulations were heated to a set temperature of 120° C. and titrated using the Coulometer until no water is remaining in the sample. 10 mg of each powder was weighed and the samples assessed. The results are shown in FIG. 5 (for 0.5 mg doses) and FIG. 6 (for 5 mg doses).

The binding ELISA method quantitatively measures the binding activity of CDP7766 to recombinant human interleukin-13 (rhIL-13). ELISA plates were coated with a solution of rhIL-13 before a blocking step is performed to avoid non-specific binding to the plates. The plates were then incubated with the HRP-conjugated detection antibody and visualized by the addition of TMB substrate. The assay is stopped by the addition of 2M sulphuric acid and the absorbance is read with a spectrophotometer at 450 nm. The relative potency (RP) of the sample is estimated by comparison to the standard curve. The results are shown in FIG. 5 (for 0.5 mg doses) and FIG. 6 (for 5 mg doses).

The VR942 formulated compositions manufactured under a controlled process and environmental conditions and stored in unit dose blisters (thereby protected from environmental conditions) demonstrated room temperature stability for up to 24 months in respect of particle size and protein stability.

### Example 2—Demographics

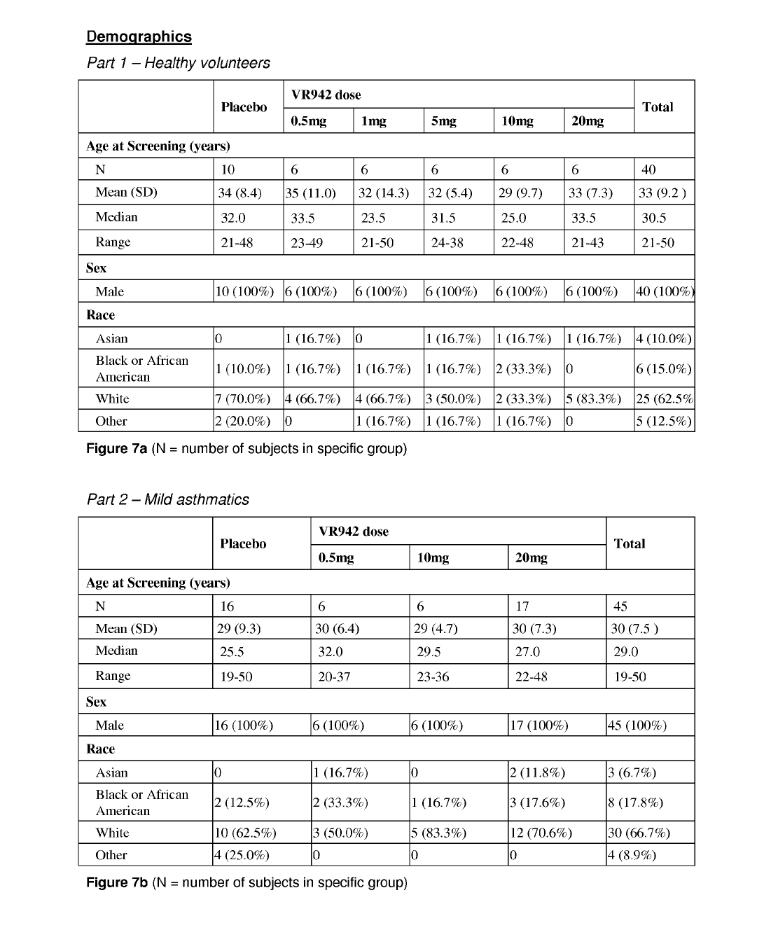

The demographic data for the Safety Population are presented in FIG. 7a, for Part 1, healthy volunteers and FIG. 7b, for Part 2, mild asthmatics. All randomised subjects were male, with a mean age of 33±7.3 years in Part 1 and 30±7.5 years in Part 2, with the majority being White (63% and 67% of subjects in Part 1 and Part 2, respectively).

Subjects had a mean height and weight of 177±6.0 cm and 77±10.6 kg and 178±5.7 cm and 78±10.6 kg in Part 1 and Part 2, respectively. Subjects were balanced between treatment groups in both parts of the study.

### Example 3—Safety

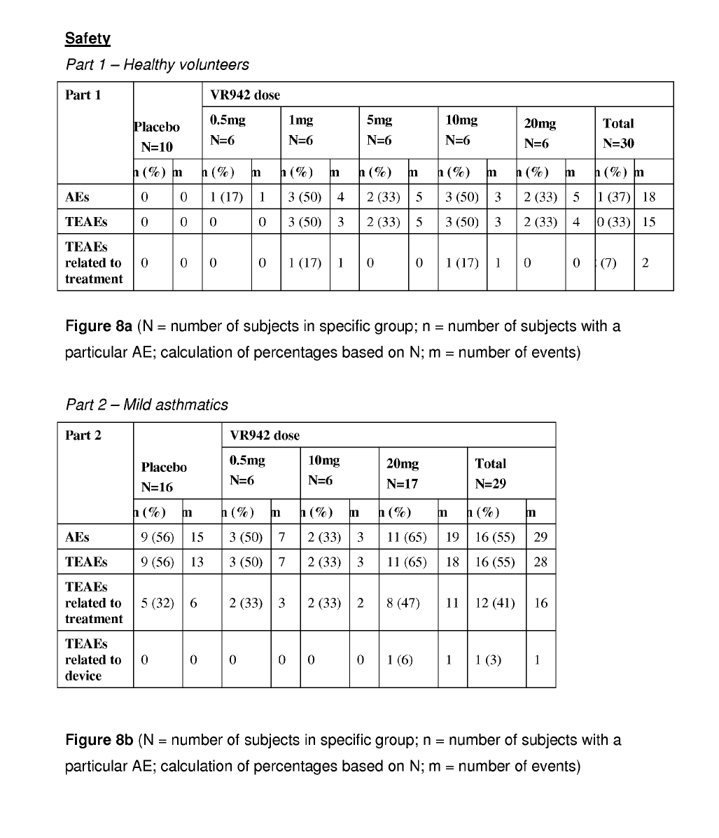

In Part 1, healthy volunteers, a total of 18 AEs were experienced by 11 subjects (37%) during the course of the study (FIG. 8a). Of these, 15 were TEAEs experienced by 10 subjects (33%). The AEs were reported by subjects across all VR942 dosing groups. Two TEAEs were considered related to study medication. No inhaler-related TEAEs, TEAEs leading to withdrawal or SAEs were reported during the study.

In Part 2, mild asthmatics, a total of 29 AEs were experienced by 16 subjects (55%) during the course of the study (FIG. 8b). All except one of these were TEAEs. The AEs were reported by subjects across all VR942 dosing groups. Sixteen TEAEs were considered to be related to study medication. No inhaler-related TEAEs, TEAEs leading to withdrawal or SAEs were reported during the study.

### Example 4—FeNO

Nitric oxide (NO) is present in virtually all mammalian organ systems, is produced by the human lung and is present in the exhaled breath of all humans. NO is recognized to play key roles in virtually all aspects of lung biology and has been implicated in the pathophysiology of lung diseases such as asthma. Patients with asthma have high levels of NO in their exhaled breath and high levels of inducible nitric oxide synthase (NOS2) enzyme expression in the epithelial cells of their airways (NO is produced by the enzyme NO synthase, which is under direct control of IL-13). The field of exhaled NO measurement has developed over the last 20 years and patients with asthma have been found to have high FeNO levels in their exhaled breath that decreased in response to treatment with corticosteroids. This quickly prompted the evaluation of FeNO as a potential non-invasive method to diagnose asthma and monitor the response to anti-inflammatory therapy. Without wishing to be bound by theory it is believed that lowering levels of airway IL-13 results in a reduction in FeNO levels which potentially leads to a reduction in airway inflammation.

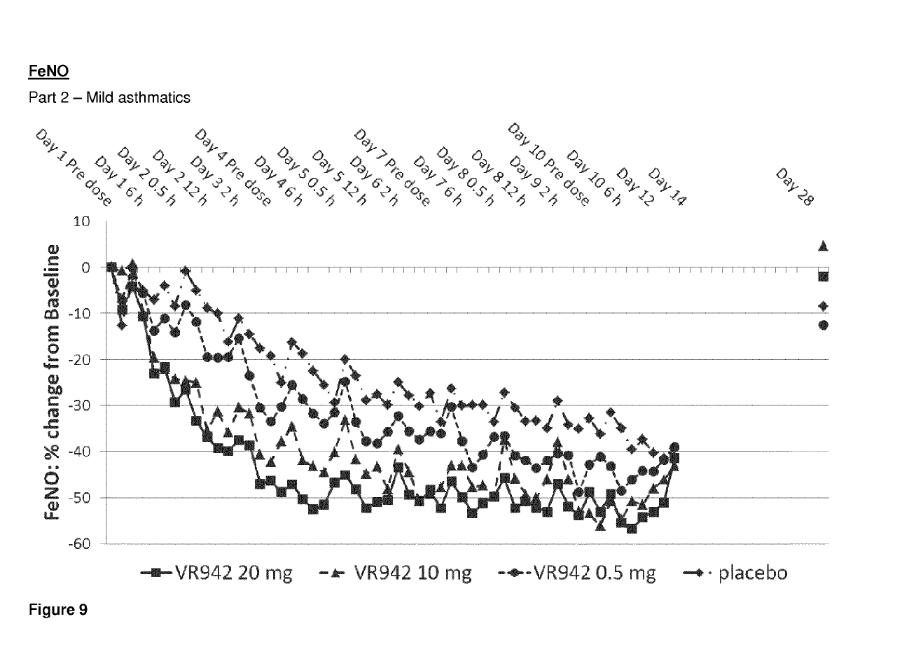

During the VR942/1/001 Phase 1 clinical study FeNO was measured before dosing and at specified time points up to 28 days following the subject's first dose. A summary of the FeNO data over the course of the study is presented for placebo and the active treatment groups in, FIGS. 9, 10, 11 14 and 15.

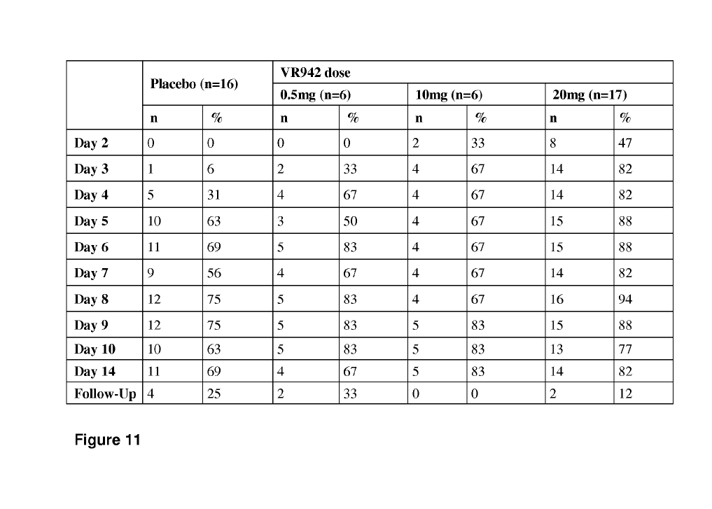

All FeNO responders are summarised in FIG. 11. At days 2, 3 and 4 a greater proportion of patients receiving 0.5 mg, 10 mg and 20 mg achieved relevant FeNO reductions of at least 30% compared to placebo. Similarly at 2 hours post-dose on Day 10, five (83%) subjects in the 0.5 mg and 10 mg VR942 groups had responded and similarly, 13 (82%) subjects in the 20 mg VR942 group. This compares to 11 (69%) of subjects in the placebo group. These levels were sustained to Day 14 (96 hours post-dose).

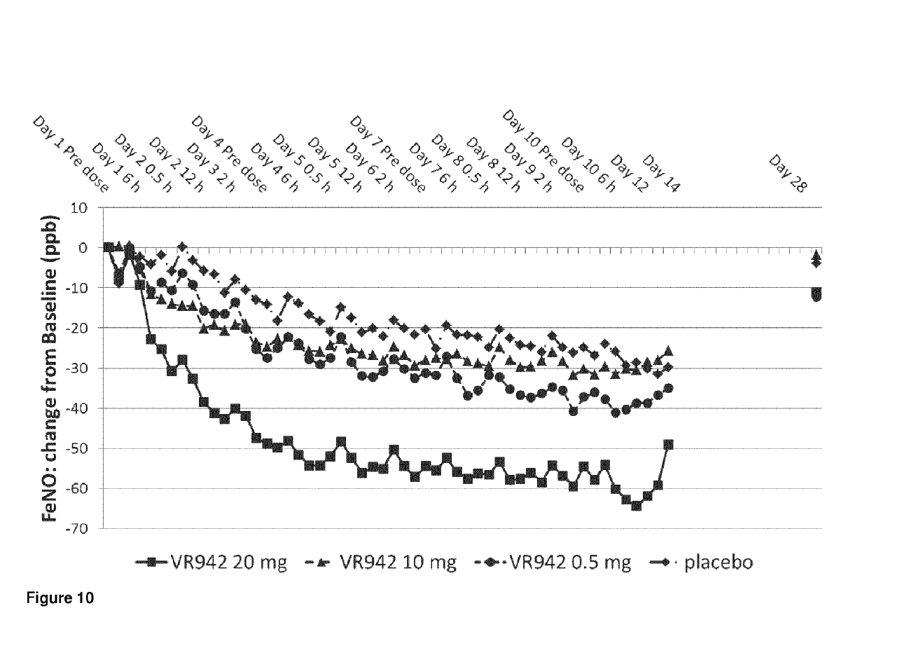

Interestingly, there was a steady reduction in mean FeNO levels from baseline over the 10-day treatment period in all active treatment groups before returning to baseline levels by the follow-up visit. For example, the percentage (%) reduction from baseline in FeNO in all the treatment groups examined was from about 13% to about 65% after 10 days of treatment (see FIGS. 9 and 14). The absolute (ppb) reduction from baseline in FeNO in all the treatment groups examined was from about 14 ppb to about 65 ppb after 10 days of treatment (see FIGS. 10 and 15).

Furthermore, the rapid, durable, and dose related reduction of FeNO versus placebo that was observed in the mild asthmatic subjects was also maintained for at least 4 days following the last administered treatment dose. Statistically significant (p<0.05) changes were reported for treatment groups subjected to 10 mg and 20 mg daily nominal doses.

### Example 5—FEV₁

Pulmonary function tests (PFTs) can assist in determining if an obstructive or restrictive disease is present in a human subject. The term PFT encompasses three different measures of lung function: spirometry, lung volumes, and diffusion capacity. PFTs are interpreted by comparing a patient value to the predicted value of a healthy subject with similar age, weight, and height. A longitudinal reduction in lung function, and particularly forced expiratory volume in one second (FEV₁), can indicate a worsening of asthma. Consequently, there is a continuing focus on the development of pharmacological interventions with the potential to improve FEV₁ outcomes.

A Phase 2 clinical study (Noonan et al. J Allergy Clin Immunol 2013) evaluated the clinical efficacy of lebrikizumab, an anti-IL-13 monoclonal antibody, in 212 asthmatics. The use of inhaled, parenteral or oral corticosteroid therapy was prohibited. The primary study end point was the relative change in pre-bronchodilator FEV₁ from baseline to the end of the treatment period at Week 12. No statistically or clinically relevant changes in FEV₁, relative to placebo, were observed and it was concluded that the blocking IL-13, a single cytokine, in this population of asthmatic patients is insufficient to improve lung function.

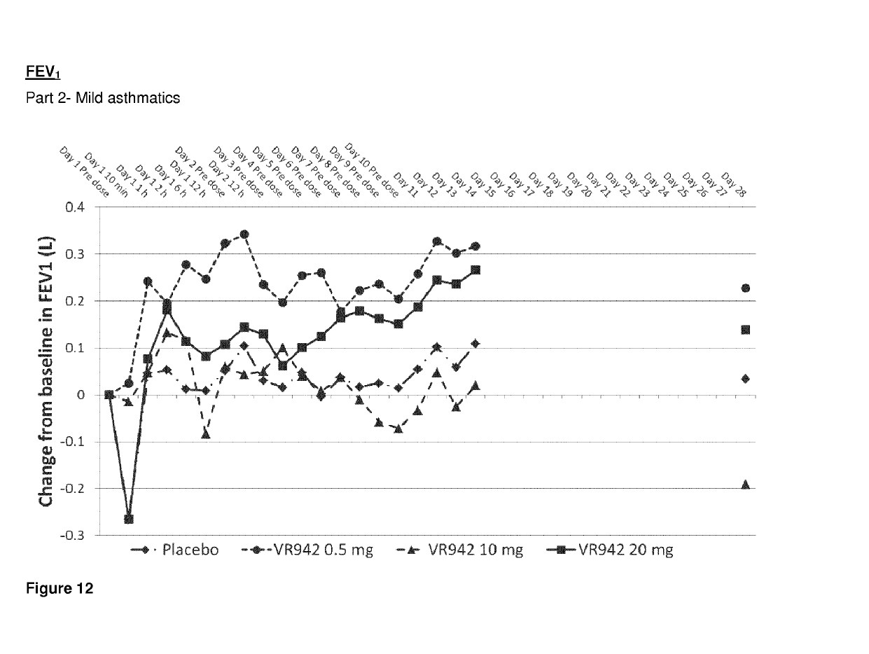

VR942 treatment over a significantly shorter time period (10-14 days) resulted in numerically and clinically relevant improvements in FEV₁ for 0.5 and 20 mg doses of VR942 (FIGS. 12 and 16).

After a 14-day no treatment period patients returned to the clinic and underwent further lung function assessment. The recorded values for all dose groups including placebo were lower than recorded at Day 14 with the greatest reductions noted for active treatment groups.

Note that analysis of Day 1 serial FEV₁ outcomes indicates an acute effect with a dose-dependent improvement in FEV₁ at 6 hours post dose. The outcome time course is more indicative of a bronchodilation response which would be unexpected of an anti-inflammatory agent.

### Example 6—Immunogenicity

In both Part 1 and Part 2 of the study, the proportion of subjects in each treatment group that tested positive for antibodies against CDP7766 was examined. Blood samples for immunogenicity assessment were taken before, and at specified time points up to 28 days following the subject's first dose.

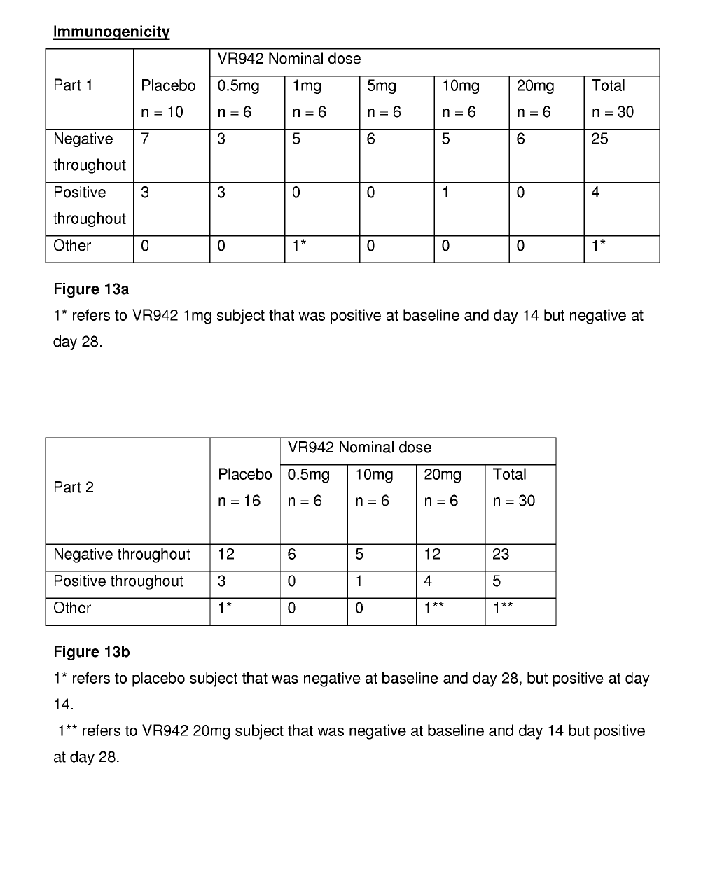

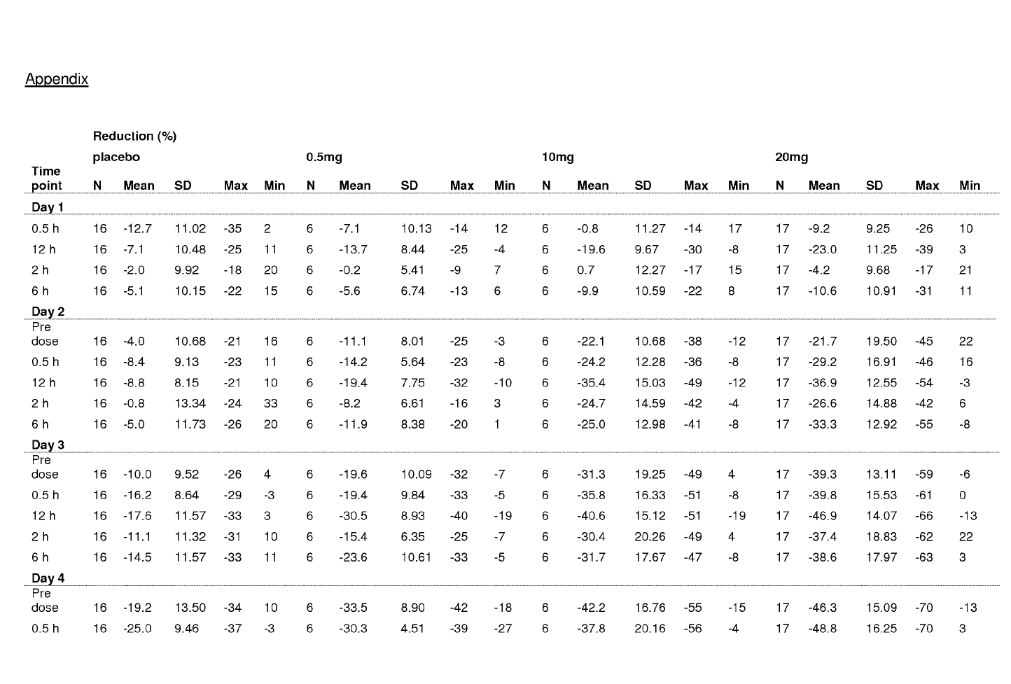

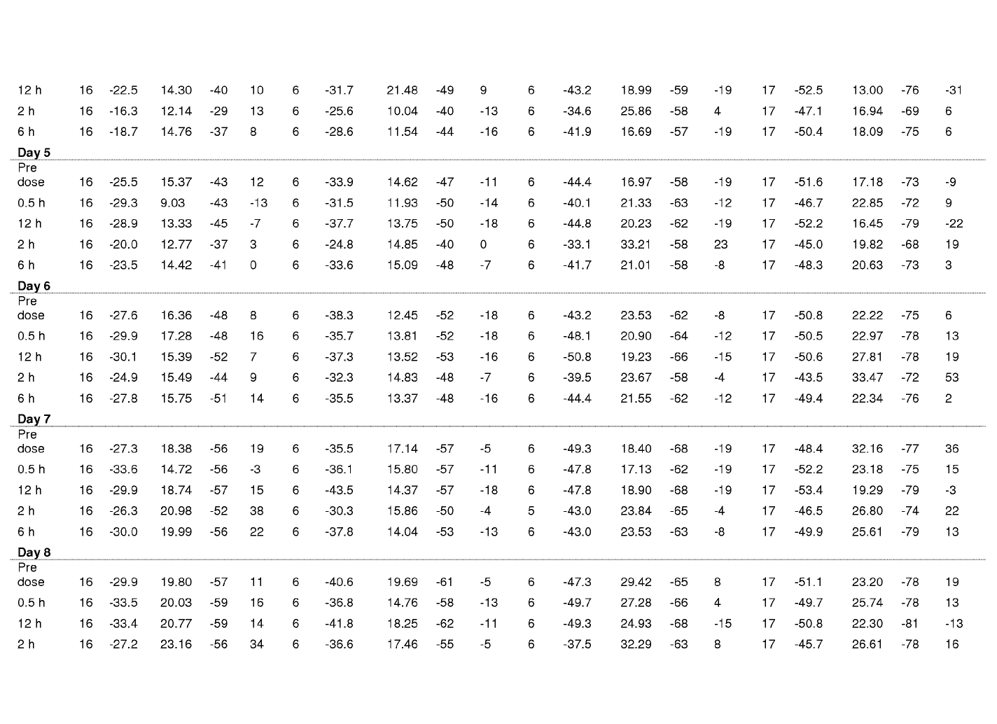

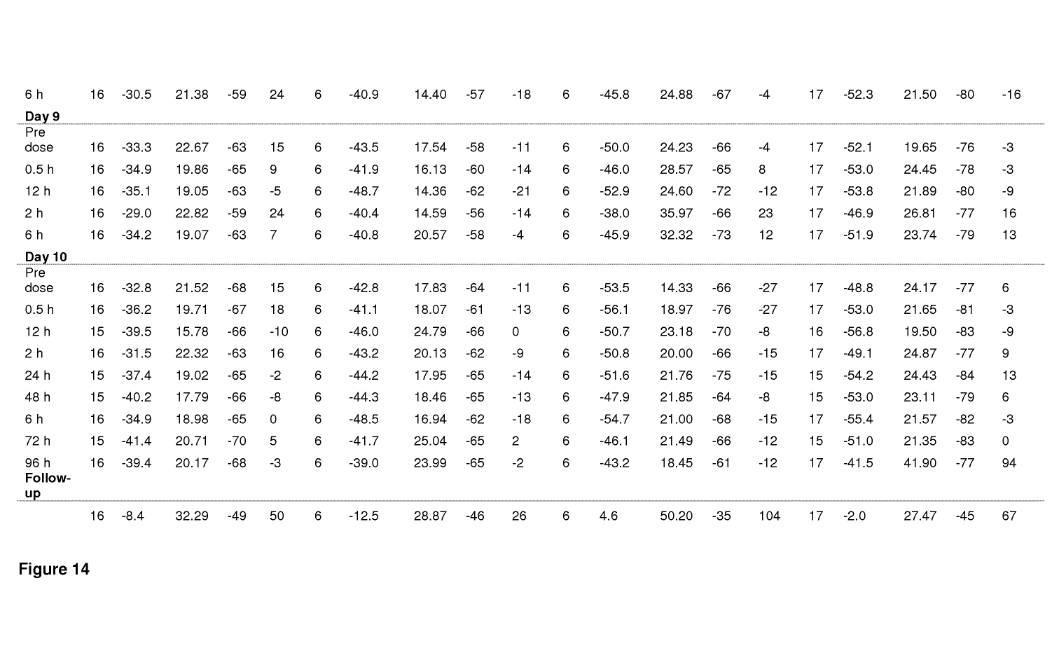

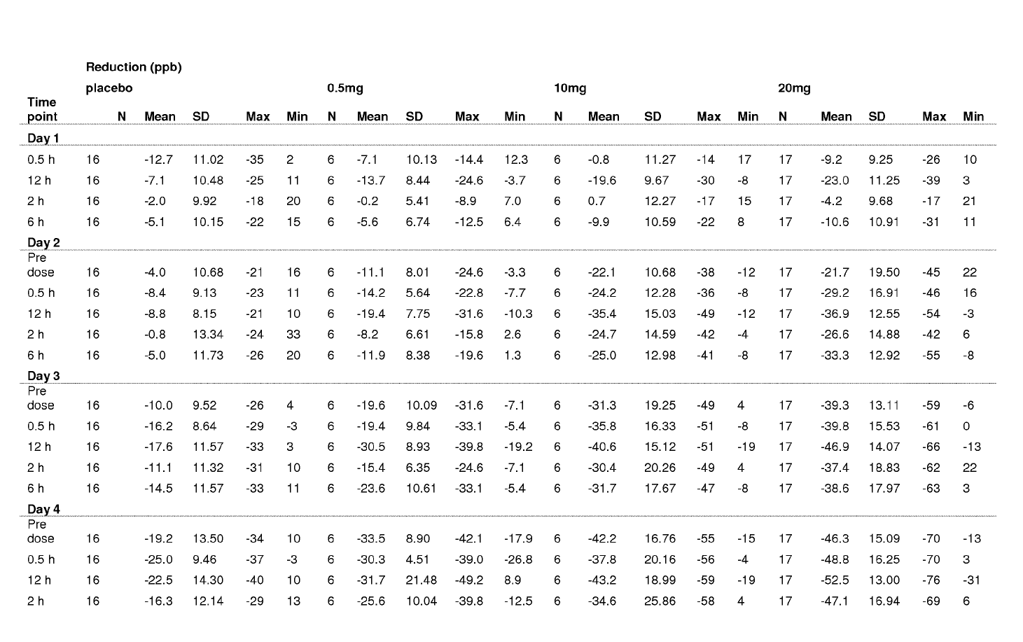

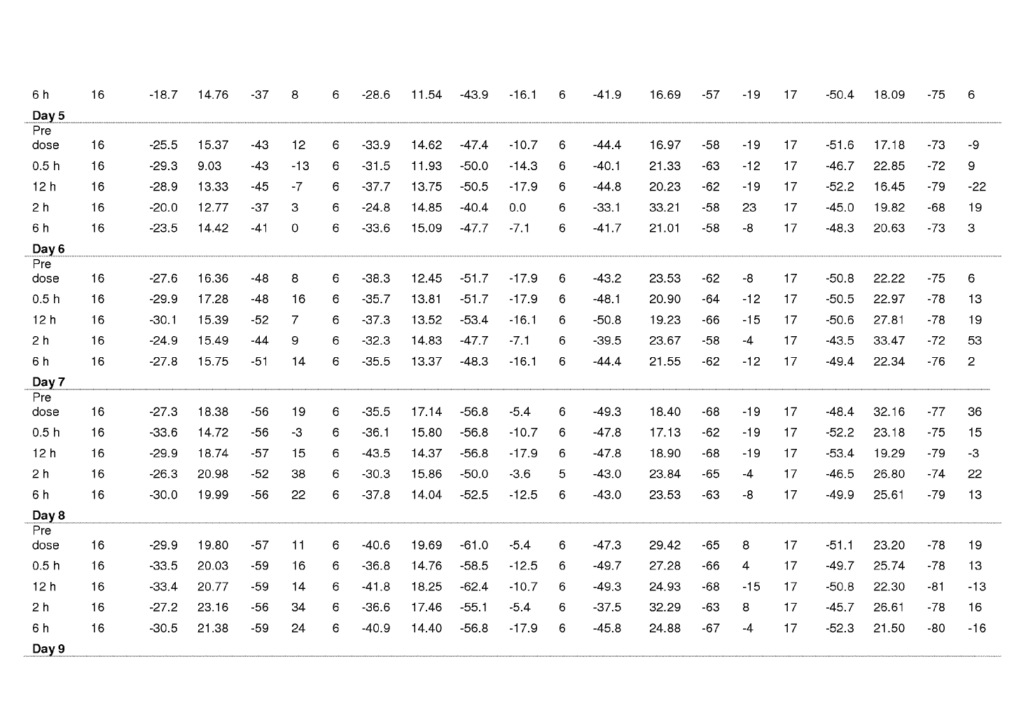

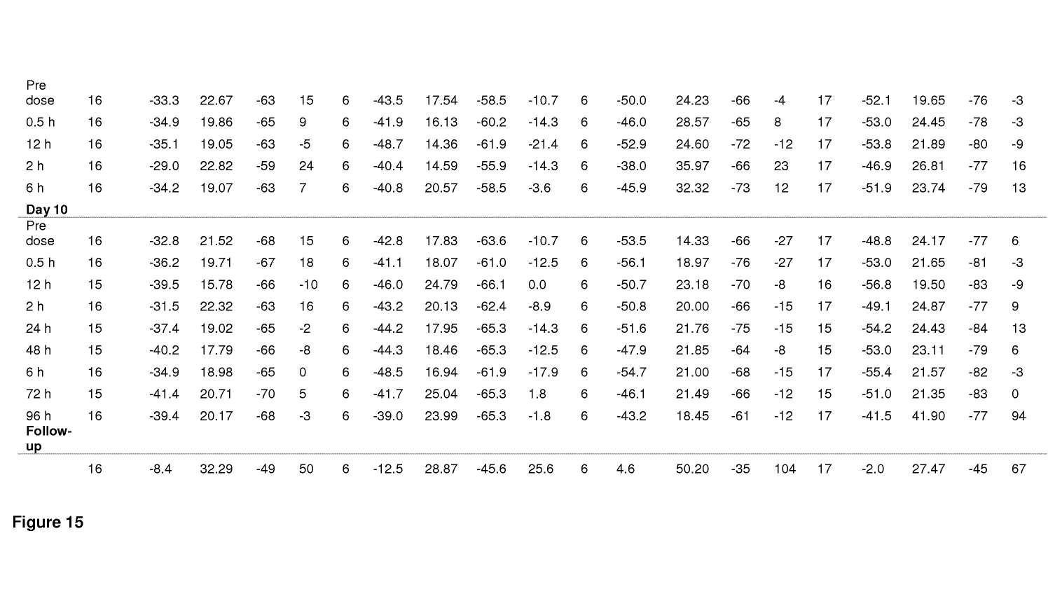

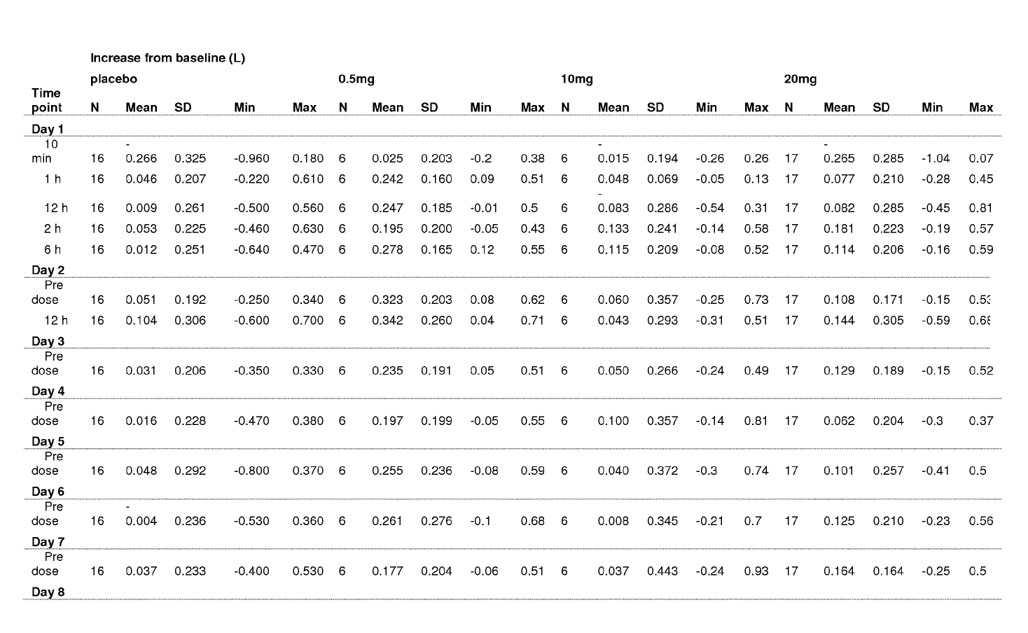

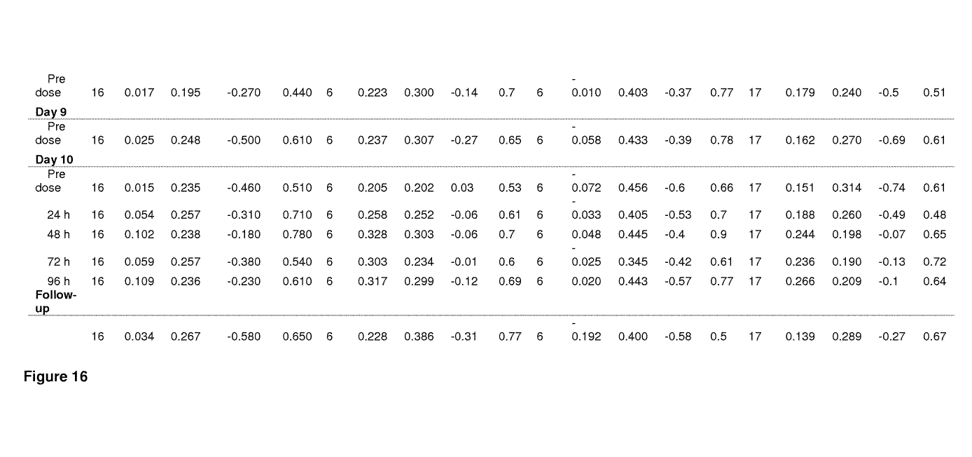

In Part 1, healthy volunteers, six of the 40 subjects tested positive for CDP7766 antibodies (determined when either both the day 14 and day 28 confirmatory statuses are positive or if only the day 28 confirmatory status is positive). However, all tested positive pre-dose and no subsequent sample showed a dilution of 2-fold or more (see FIG. 13a).

In Part 2, mild asthmatics, nine of the 45 subjects tested positive for CDP7766 antibodies. No subject in the VR942 0.5 mg group tested positive. All those in the placebo group and VR942 10 mg group tested positive pre-dose and no subsequent sample showed a dilution of 2-fold or more. However, one of the five subjects who tested positive at day 28, was negative pre-dose and at day 14 (see FIG. 13b).

### Example 7—PK

Pharmacokinetic analysis was conducted during clinical study VR942/1/001 to determine how much drug substance (CDP7766) reached the systemic circulation after the administration of single doses to healthy volunteers and repeated single daily doses to mild asthmatics over a 10-day in-clinic treatment period. Analysis of blood samples collected from patients randomised to the highest study dose revealed that all CDP7766 concentrations were below the assay lower limit of quantification (99.6 ng/ml).

1. An inhalable powder composition comprising a) an antagonistic antibody which binds human IL-13, b) leucine and c) trehalose.

2. Inhalable particles comprising a) an antagonistic antibody which binds human IL-13, b) leucine and c) trehalose.

3. An inhalable powder composition as claimed in claim 1, wherein the antibody is selected from the group consisting of: a complete antibody molecule having full length heavy and light chains or a fragment thereof, such as a Fab, modified Fab′, Fab′, F(ab′)₂, Fv, VH, VL or scFv fragment.

4. An inhalable powder composition as claimed in claim 1, wherein the antibody comprises a heavy chain, wherein the variable domain of the heavy chain comprises the sequence given in SEQ ID NO:3 and, additionally comprises a light chain, wherein the variable domain of the light chain comprises the sequence given in SEQ ID NO:1.

5. An inhalable powder composition as claimed in claim 1, wherein the antibody is CDP7766.

6. An inhalable powder composition as claimed in claim 1, wherein the antibody comprises a light chain that has at least 60% homology, identity or similarity to the sequence given in SEQ ID NO:1, or at least 70%, at least 80%, at least 90%, at least 95% or at least 98% homology, identity or similarity.

7. An inhalable powder composition as claimed in claim 1, wherein the antibody comprises a heavy chain that has at least 60% homology, identity or similarity to the sequence given in SEQ ID NO:3, or at least 70%, at least 80%, at least 90%, at least 95% or at least 98% homology, identity or similarity.

8. An inhalable powder composition as claimed in claim 1, wherein the antibody is present in an amount less than or equal to about 40% by weight of the dry weight of the powder composition, or less than or equal to about 30%, or less than or equal to about 20%, or less than or equal to about 10% or less than or equal to about 4%, or less than or equal to about 3%, or less than or equal to about 2%, or less than or equal to about 1% or less than or equal to about 0.5%.

9. An inhalable powder composition as claimed in claim 1, wherein the antibody is present in an amount of from about 0.5% to about 40%, or about 1% to about 40%, or about 2% to about 40% or 3% to about 40% or about 4% to about 40% by weight of the dry weight of the composition.

10. An inhalable powder composition as claimed in claim 1, wherein the antibody is present in an amount of from about 10% to about 40% or about 20% to about 40% or about 30% to about 40% by weight of the dry weight of the composition.

11. An inhalable powder composition as claimed in claim 1, wherein the leucine is present in an amount less than or equal to about 25% by weight of the dry weight of the powder composition, or less than or equal to about 20%, less than or equal to about 15%, or less than or equal to about 10%, or less than or equal to about 5%.

12. An inhalable powder composition as claimed in claim 1, wherein the leucine is present in an amount of from about 5% to about 25% by weight of the dry weight of the powder composition, or from about 10% to about 20% by weight of the dry weight of the powder composition.

13. An inhalable powder composition as claimed in claim 1, wherein the antibody is present in less than or equal to about 40% by weight of the dry weight of the powder composition and the leucine is present in less than or equal to about 20%, or less than or equal to 15% or less than or equal to about 10% by weight of the dry weight of the powder composition, and typically more than 5% by weight of the dry weight of the powder composition.

14. An inhalable powder composition as claimed in claim 1, wherein the antibody is present in less than or equal to about 30% by weight of the dry weight of the powder composition and the leucine is present in less than or equal to about 20%, or less than or equal to 15%, or less than or equal to about 10% by weight of the dry weight of the powder composition, and typically more than 5% by weight of the dry weight of the powder composition.

15. An inhalable powder composition as claimed in claim 1, wherein the antibody is present in less than or equal to about 20% by weight of the dry weight of the powder composition and the leucine is present in less than or equal to about 20%, or less than or equal to 15%, or less than or equal to about 10% by weight of the dry weight of the powder composition, and typically more than 5% by weight of the dry weight of the powder composition.

16. An inhalable powder composition as claimed in claim 1, wherein the antibody is present in less than or equal to about 4% by weight of the dry weight of the powder composition and the leucine is present in less than or equal to about 20%, or less than or equal to 15% or less than or equal to about 10% by weight of the dry weight of the powder composition, and typically more than 5% by weight of the dry weight of the powder composition.

17. An inhalable powder composition as claimed in claim 1, wherein the antibody is present in an amount of from about 4 to about 40% by weight of the dry weight of the composition and the leucine is present in an amount of from about 10% to about 20% by weight of the dry weight of the composition

18. An inhalable powder composition as claimed in claim 1, wherein the trehalose is present in an amount less than or equal to about 90% by weight of the dry weight of the powder composition, or less than or equal to about 80%, less than or equal to about 75%, less than or equal to about 70%, less than or equal to about 65%, less than or equal to about 60%, less than or equal to about 55%, less than or equal to about 50%, less than or equal to about 45%, or less than or equal to about 40%, or less than or equal to about 30% or less than or equal to about 20%.

19. An inhalable powder composition as claimed in claim 1, wherein the trehalose is present in an amount of from about 40% to about 90% by weight of the dry weight of the composition, or from about 55% to about 65% by weight of the dry weight of the composition.

20. An inhalable powder composition as claimed in claim 1, wherein the antibody is present in an amount less than or equal to about 40% by weight of the dry weight of the powder composition, the leucine is present in an amount of about 10% or from about 5% to about 25% by weight of the dry weight of the powder composition and the trehalose is present in an amount of about 45% or from about 35% to about 50% by weight of the dry weight of the powder composition.

21. An inhalable powder composition as claimed in claim 1, wherein the antibody is present in less than or equal to about 30% by weight of the dry weight of the powder composition and the leucine is present in an amount of about 10% or from about 5% to about 25% by weight of the dry weight of the powder composition and the trehalose is present in an amount of about 35% or from about 25% to about 40% by weight of the dry weight of the powder composition.

22. An inhalable powder composition as claimed in claim 1, wherein the antibody is present in less than or equal to about 20% by weight of the dry weight of the powder composition and the leucine is present in an amount of about 10% or from about 5% to about 25% by weight of the dry weight of the powder composition and the trehalose is present in an amount of about 67% or from 57% to about 70% by weight of the dry weight of the powder composition.

23. An inhalable powder composition as claimed in claim 1, wherein the antibody is present in less than or equal to about 4% by weight of the dry weight of the powder composition and the leucine is present in an amount of about 10% or from about 5% to about 25% by weight of the dry weight of the powder composition and the trehalose is present in an amount of about 85% or from about 75% to about 90% by weight of the dry weight of the powder composition.

24. An inhalable powder composition as claimed in claim 1, wherein the trehalose is present as amorphous trehalose.

25. An inhalable powder composition as claimed in claim 1, wherein the composition further comprises buffer salts such as NaCl and sodium phosphate (NaH₂PO₄).

26. An inhalable powder composition as claimed in claim 1, wherein the total buffer salts are present in an amount of less than or equal to about 7.5% by weight of the dry weight of the powder composition, or less than or equal to about 6% or less than or equal to about 5.3% or less than or equal to about 4% or less than or equal to about 3% or less than or equal to about 2.7% or less than or equal to about 2% or less than or equal to about 1% or less than or equal to about 0.5% by weight of the dry weight of the powder composition.

27. An inhalable powder composition as claimed in claim 1, wherein the total buffer salts are present in an amount of from about 0.5 to about 7.5% or from about 0.5 to about 5.3% by weight of the dry weight of the powder composition.

28. An inhalable powder composition as claimed in claim 1, wherein, the composition further comprises an inhalable corticosteroid and/or a long-acting beta 2-agonist.

29. An inhalable powder composition as claimed in claim 1, wherein the particle size distribution (PSD) of d₉₀ is less than or equal to about 10 μm, or less than or equal to about 9.5 μm, or less than or equal to about 9 μm, or less than or equal to about 8.5 μm, or less than or equal to about 8 μm, or less than or equal to about 7.5 μm, or less than or equal to about 7 μm.

30. An inhalable powder composition as claimed in claim 1, wherein the PSD of d₉₀ is from about 3 μm to about 8 μm or from about 4 μm to about 8 μm or from about 4 μm to about 7 μm.

31. An inhalable powder composition as claimed in claim 1, wherein the PSD of d₉₀ remains less than 8 μm after 1 m or 2 m or 3 m or 6 m of storage at either 25° C./60% RH or 30° C./65% RH or 40° C./75% RH.

32. An inhalable powder composition as claimed in claim 1, which has a tap density of less than or equal to about 0.7 g/cm³, or less than or equal to about 0.62 g/cm³ or less than or equal to about 0.61 g/cm³, or less than or equal to about 0.60 g/cm³ or less than or equal to about 0.59 g/cm³ or less than or equal to about 0.58 g/cm³, or less than or equal to about 0.57 g/cm³.

33. An inhalable powder composition as claimed in claim 1, which has a tap density of from about 0.4 g/cm³ to about 0.7 g/cm³, or from about 0.55 g/cm³ to about 0.65 g/cm³.

34. An inhalable powder composition as claimed in claim 1, which has a moisture content of less than or equal to about 5%, or less than or equal to about 4%, or less than or equal to about 3%, or less than or equal to about 2% or less than or equal to about 1% by weight of the dry weight of the powder composition.

35. An inhalable powder composition as claimed in claim 1, which has a moisture content of from about 1% to about 5% or from about 2% to about 4% by weight of the dry weight of the powder composition.

36. An inhalable powder composition as claimed in claim 1, which has a moisture content of from about 2% to about 4% by weight of the dry weight of the powder composition after 1 m or 2 m or 3 m or 6 m of storage at either 25° C./60% RH or 30° C./65% RH or 40° C./75% RH.

37. An inhalable powder composition as claimed in claim 1, which has a glass transition temperature (T_{g}) equal to or greater than 60° C.

38. An inhalable powder composition as claimed in claim 1, which has a T_{g} of from about 60° C. to about 95° C. or from about 65° C. to about 90° C. or from 65° C. to about 85° C. or from 65° C. to about 75° C.

39. An inhalable powder composition as claimed in claim 1, which has a T_{g} of from about 60° C. to about 95° C. after 1 m or 2 m or 3 m or 6 m of storage at either 25° C./60% RH or 30° C./65% RH or 40° C./75% RH.

40. A process for preparing an inhalable dry powder composition, the process comprising the steps of;
(i) preparing a first aqueous solution and/or suspension comprising leucine and trehalose;
(ii) preparing a second aqueous solution and/or suspension comprising an antagonistic antibody which binds human IL-13 and buffer salt;
(iii) mixing the first and second aqueous solutions and/or suspensions from steps (i) and (ii) to form a feedstock solution and/or suspension; and
(iv) spray-drying the feedstock solution and/or suspension from step (iii).

41. A process as claimed in claim 40, wherein the second aqueous solution comprises buffer salt.

42. A process as claimed in claim 41, wherein the buffer salt is PBS.

43. A process as claimed in claim 40, wherein the total solids content of the feedstock solution and/or suspension is from about 3.5 to 5.5% w/v.

44. Spray-dried inhalable particles comprising a) an antagonistic antibody which binds human IL-13, b) leucine and c) trehalose, the particles being obtainable by the process of claim 40.

45. A container comprising an inhalable powder composition as claimed in claim 1.

46. The container according to claim 45, wherein the container is a blister such as a unit dose foil blister.

47. A dry powder inhaler comprising an inhalable powder composition as claimed in claim 1.

48. The inhaler according to claim 47, wherein the inhaler is a passive unit dose inhaler.

49. A pharmaceutical kit comprising:
(i) an inhalable powder composition as claimed in claim 1, and
(ii) a dry powder inhaler.

50-51. (canceled)

52. A method of treatment of asthma in a subject suffering from or susceptible to that condition, which method comprises the administration to the subject of an inhalable powder composition as claimed in claim 1.

53. A method as claimed in claim 52, wherein the subject is an adult.

54. Inhalable particles as claimed in claim 2, wherein the antibody is selected from the group consisting of: a complete antibody molecule having full length heavy and light chains or a fragment thereof, such as a Fab, modified Fab′, Fab′, F(ab′)₂, Fv, VH, VL or scFv fragment.

55. Inhalable particles as claimed in claim 2, wherein the antibody comprises a heavy chain, wherein the variable domain of the heavy chain comprises the sequence given in SEQ ID NO:3 and, additionally comprises a light chain, wherein the variable domain of the light chain comprises the sequence given in SEQ ID NO:1.

56. Inhalable particles as claimed in claim 2, wherein the antibody is CDP7766.

57. Inhalable particles as claimed in claim 2, wherein the antibody comprises a light chain that has at least 60% homology, identity or similarity to the sequence given in SEQ ID NO:1, or at least 70%, at least 80%, at least 90%, at least 95% or at least 98% homology, identity or similarity.

58. Inhalable particles as claimed in claim 2, wherein the antibody comprises a heavy chain that has at least 60% homology, identity or similarity to the sequence given in SEQ ID NO:3, or at least 70%, at least 80%, at least 90%, at least 95% or at least 98% homology, identity or similarity.

59. Inhalable particles as claimed in claim 2, wherein the antibody is present in an amount less than or equal to about 40% by weight of the dry weight of the powder composition, or less than or equal to about 30%, or less than or equal to about 20%, or less than or equal to about 10% or less than or equal to about 4%, or less than or equal to about 3%, or less than or equal to about 2%, or less than or equal to about 1% or less than or equal to about 0.5%.

60. Inhalable particles as claimed in claim 2, wherein the antibody is present in an amount of from about 0.5% to about 40%, or about 1% to about 40%, or about 2% to about 40% or 3% to about 40% or about 4% to about 40% by weight of the dry weight of the composition.

61. Inhalable particles as claimed in claim 2, wherein the antibody is present in an amount of from about 10% to about 40% or about 20% to about 40% or about 30% to about 40% by weight of the dry weight of the composition.

62. Inhalable particles as claimed in claim 2, wherein the leucine is present in an amount less than or equal to about 25% by weight of the dry weight of the powder composition, or less than or equal to about 20%, less than or equal to about 15%, or less than or equal to about 10%, or less than or equal to about 5%.

63. Inhalable particles as claimed in claim 2, wherein the leucine is present in an amount of from about 5% to about 25% by weight of the dry weight of the powder composition, or from about 10% to about 20% by weight of the dry weight of the powder composition.

64. Inhalable particles as claimed in claim 2, wherein the antibody is present in less than or equal to about 40% by weight of the dry weight of the powder composition and the leucine is present in less than or equal to about 20%, or less than or equal to 15% or less than or equal to about 10% by weight of the dry weight of the powder composition, and typically more than 5% by weight of the dry weight of the powder composition.

65. Inhalable particles as claimed in claim 2, wherein the antibody is present in less than or equal to about 30% by weight of the dry weight of the powder composition and the leucine is present in less than or equal to about 20%, or less than or equal to 15%, or less than or equal to about 10% by weight of the dry weight of the powder composition, and typically more than 5% by weight of the dry weight of the powder composition.

66. Inhalable particles as claimed in claim 2, wherein the antibody is present in less than or equal to about 20% by weight of the dry weight of the powder composition and the leucine is present in less than or equal to about 20%, or less than or equal to 15%, or less than or equal to about 10% by weight of the dry weight of the powder composition, and typically more than 5% by weight of the dry weight of the powder composition.

67. Inhalable particles as claimed in claim 2, wherein the antibody is present in less than or equal to about 4% by weight of the dry weight of the powder composition and the leucine is present in less than or equal to about 20%, or less than or equal to 15% or less than or equal to about 10% by weight of the dry weight of the powder composition, and typically more than 5% by weight of the dry weight of the powder composition.

68. Inhalable particles as claimed in claim 2, wherein the antibody is present in an amount of from about 4 to about 40% by weight of the dry weight of the composition and the leucine is present in an amount of from about 10% to about 20% by weight of the dry weight of the composition

69. Inhalable particles as claimed in claim 2, wherein the trehalose is present in an amount less than or equal to about 90% by weight of the dry weight of the powder composition, or less than or equal to about 80%, less than or equal to about 75%, less than or equal to about 70%, less than or equal to about 65%, less than or equal to about 60%, less than or equal to about 55%, less than or equal to about 50%, less than or equal to about 45%, or less than or equal to about 40%, or less than or equal to about 30% or less than or equal to about 20%.

70. Inhalable particles as claimed in claim 2, wherein the trehalose is present in an amount of from about 40% to about 90% by weight of the dry weight of the composition, or from about 55% to about 65% by weight of the dry weight of the composition.

71. Inhalable particles as claimed in claim 2, wherein the antibody is present in an amount less than or equal to about 40% by weight of the dry weight of the powder composition, the leucine is present in an amount of about 10% or from about 5% to about 25% by weight of the dry weight of the powder composition and the trehalose is present in an amount of about 45% or from about 35% to about 50% by weight of the dry weight of the powder composition.

72. Inhalable particles as claimed in claim 2, wherein the antibody is present in less than or equal to about 30% by weight of the dry weight of the powder composition and the leucine is present in an amount of about 10% or from about 5% to about 25% by weight of the dry weight of the powder composition and the trehalose is present in an amount of about 35% or from about 25% to about 40% by weight of the dry weight of the powder composition.

73. Inhalable particles as claimed in claim 2, wherein the antibody is present in less than or equal to about 20% by weight of the dry weight of the powder composition and the leucine is present in an amount of about 10% or from about 5% to about 25% by weight of the dry weight of the powder composition and the trehalose is present in an amount of about 67% or from 57% to about 70% by weight of the dry weight of the powder composition.

74. Inhalable particles as claimed in claim 2, wherein the antibody is present in less than or equal to about 4% by weight of the dry weight of the powder composition and the leucine is present in an amount of about 10% or from about 5% to about 25% by weight of the dry weight of the powder composition and the trehalose is present in an amount of about 85% or from about 75% to about 90% by weight of the dry weight of the powder composition.

75. Inhalable particles as claimed in claim 2, wherein the trehalose is present as amorphous trehalose.

76. Inhalable particles as claimed in claim 2, wherein the composition further comprises buffer salts such as NaCl and sodium phosphate (NaH₂PO₄).

77. Inhalable particles as claimed in claim 2, wherein the total buffer salts are present in an amount of less than or equal to about 7.5% by weight of the dry weight of the powder composition, or less than or equal to about 6% or less than or equal to about 5.3% or less than or equal to about 4% or less than or equal to about 3% or less than or equal to about 2.7% or less than or equal to about 2% or less than or equal to about 1% or less than or equal to about 0.5% by weight of the dry weight of the powder composition.

78. Inhalable particles as claimed in claim 2, wherein the total buffer salts are present in an amount of from about 0.5 to about 7.5% or from about 0.5 to about 5.3% by weight of the dry weight of the powder composition.

79. Inhalable particles as claimed in claim 2, wherein, the composition further comprises an inhalable corticosteroid and/or a long-acting beta 2-agonist.

80. Inhalable particles as claimed in claim 2, wherein the particle size distribution (PSD) of d₉₀ is less than or equal to about 10 μm, or less than or equal to about 9.5μm, or less than or equal to about 9 μm, or less than or equal to about 8.5 μm, or less than or equal to about 8 μm, or less than or equal to about 7.5 μm, or less than or equal to about 7 μm.

81. Inhalable particles as claimed in claim 2, wherein the PSD of d₉₀ is from about 3 μm to about 8 μm or from about 4 μm to about 8 μm or from about 4 μm to about 7 μm.

82. Inhalable particles as claimed in claim 2, wherein the PSD of d₉₀ remains less than 8 μm after 1 m or 2 m or 3 m or 6 m of storage at either 25° C./60% RH or 30° C./65% RH or 40° C./75% RH.
